(19) United States
(12) Patent Application Publication
(10) Pub. No.: US 2003/0022343 A1
(43) Pub. Date: Jan. 30, 2003

(54) Inositolphosphoryl ceramide (IPC) synthase genes from fungi

(76) Inventors: Steven Alan Heidler, Fishers, IN (US); Jeffrey Alan Radding, Carmel, IN (US)

Correspondence Address: Tina M. Tucker, Eli Lilly and Company, Lilly Corporate Center, Patent Division DC: 1104, Indianapolis, IN, 46285, US

(21) Appl. No.: 09/742,581

(22) Filed: Dec. 20, 2000

Related U.S. Application Data
(62) Division of application No. 09/041,075, filed on Mar. 10, 1998, now Pat. No. H002022.
(60) Provisional application No. 60/043,591, filed on Apr. 15, 1997.
(60) Provisional application No. 60/044,095, filed on Apr. 22, 1997.
(60) Provisional application No. 60/046,348, filed on May 13, 1997.
(60) Provisional application No. 60/053,320, filed on Jul. 21, 1997.
(60) Provisional application No. 60/062,971, filed on Oct. 17, 1997.
(60) Provisional application No. 60/076,436, filed on Mar. 2, 1998.

(51) Int. Cl. C12P 21/02; C07H 21/04; C12N 9/16; C12N 1/18; C12N 15/74

(52) U.S. Cl. 435/196; 435/69.1; 435/254.2; 435/320.1; 536/23.2

(57) ABSTRACT

The invention provides isolated nucleic acid compounds encoding IPC synthase or subunit thereof from fungal cells. Also provided are vectors and transformed heterologous host cells for expressing IPC synthase and a method for identifying compounds that inhibit a fungal IPC synthase.

### BACKGROUND OF THE INVENTION

This invention relates to recombinant DNA technology. In particular the invention pertains to the isolation of novel genes and proteins that encode IPC synthase or subunit thereof from a variety of fungi and the use of said proteins in screens for inhibitors of IPC Synthase.

The incidence of life-threatening fungal infections is increasing at an alarming rate. With the exception of Staphylococci infections, the fungus C. albicans represents the fastest growing area of concern in hospital acquired bloodstream infections. About 90% of nosocomial fungal infections are caused by species of Candida with the remaining 10% being attributable to infections by Aspergillus, Cryptococcus, and Pneumocystis. While effective antifungal compounds have been developed for Candida there is growing concern that the rise in the incidence of fungal infections may portend greater resistance and virulence in the future. Moreover, anti-Candida compounds frequently do not possess clinically significant activity against other fungal species.

Inositolphosphoryl ceramides are sphingolipids found in a number of fungi including but not limited to S. cerevisiae, S. pombe, C. albicans, A. fumigatus, A. nidulans and H. capsulatum. A step of sphingolipid biosynthesis unique to fungi and plants is catalyzed by the enzyme IPC synthase. The IPC synthase step, covalently links inositol phosphate and ceramide, and is essential for viability in S. cerevisiae. Although some elements of sphingolipid biosynthesis in fungi are shared with mammalian systems, the pathways diverge at the step after formation of ceramide. Thus, the formation of inositolphosphoryl ceramide is unique to fungi and plants, making IPC synthase a good molecular target for antifungal chemotherapy.

While compounds that target IPC synthase bode well for the future of anti-fungal therapy, presently there are no clinically useful compounds that act at this step. Thus, there is a need for new compounds that inhibit IPC synthase.

### BRIEF SUMMARY OF THE INVENTION

The present invention relates to fungal IPC synthase and to screens for inhibitors thereof.

In one embodiment the invention relates to fungal genes that encode IPC synthase, or subunit thereof.

In another embodiment, the invention relates to fungal IPC synthase genes identified herein as SEQ ID NO:1, SEQ ID NO:4, SEQ ID NO:7, SEQ ID NO:10, SEQ ID NO:19, and SEQ ID NO:20.

In another embodiment, the invention relates to nucleic acids that are at least 70% homologous, and/or, will hybridize under high stringency conditions to a sequence identified herein as SEQ ID NO:1, SEQ ID NO:4, SEQ ID NO:7, SEQ ID NO:10, SEQ ID NO:19, and SEQ ID NO:20.

In another embodiment the present invention pertains to the proteins produced by IPC synthase genes.

In yet another embodiment, the invention relates to proteins designated herein as SEQ ID NO 2, SEQ ID NO 5, SEQ ID NO 8, SEQ ID NO 11, and SEQ ID NO:21.

In still another embodiment the invention relates to the use of purified fungal IPC synthase or subunit thereof in high throughput screens for inhibitors of fungal IPC synthase, said IPC synthase being designated herein as SEQ ID NO:2, SEQ ID NO:5, SEQ ID NO:8, SEQ ID NO:11, SEQ ID NO:14, SEQ ID NO:17, and SEQ ID NO:21.

In another embodiment the invention relates to the use of recombinant host cells that carry a vector that expresses a fungal IPC synthase in high throughput screens for inhibitors of fungal IPC synthase.

In another embodiment the invention relates to the use of the IPC synthase genes disclosed herein, or fragments thereof, as hybridization probes or PCR primers to identify and isolate homologous genes that are related in sequence and/or function.

### DEFINITIONS

The terms “cleavage” or “restriction” of DNA refers to the catalytic cleavage of the DNA with a restriction enzyme that acts only at certain sequences in the DNA (viz. sequence-specific endonucleases). The various restriction enzymes used herein are commercially available and their reaction conditions, cofactors, and other requirements are used in the manner well known to one of ordinary skill in the art. Appropriate buffers and substrate amounts for particular restriction enzymes are specified by the manufacturer or can readily be found in the literature.

The term “fusion protein” denotes a hybrid protein molecule not found in nature comprising a translational fusion or enzymatic fusion in which two or more different proteins or fragments thereof are covalently linked on a single polypeptide chain.

The term “plasmid” refers to an extrachromosomal genetic element. The starting plasmids herein are either commercially available, publicly available on an unrestricted basis, or can be constructed from available plasmids in accordance with published procedures. In addition, equivalent plasmids to those described are known in the art and will be apparent to the ordinarily skilled artisan.

“Recombinant DNA cloning vector” as used herein refers to any autonomously replicating agent, including, but not limited to, plasmids and phages, comprising a DNA molecule to which one or more additional DNA segments can or have been added.

The term “recombinant DNA expression vector” as used herein refers to any recombinant DNA cloning vector, for example a plasmid or phage, in which a promoter and other regulatory elements are present to enable transcription of the inserted DNA.

The term “vector” as used herein refers to a nucleic acid compound used for introducing exogenous DNA into host cells. A vector comprises a nucleotide sequence which may encode one or more protein molecules. Plasmid, cosmids, viruses, and bacteriophages, in the natural state or which have undergone recombinant engineering, are examples of commonly used vectors.

The terms “complementary” or “complementarity” as used herein refers to the capacity of purine and pyrimidine nucleotides to associate through hydrogen bonding in double stranded nucleic acid molecules. The following base pairs are complementary: guanine and cytosine; adenine and thymine; and adenine and uracil.

“Isolated nucleic acid compound” refers to any RNA or DNA sequence, however constructed or synthesized, which is locationally distinct from its natural location.

A “primer” is a nucleic acid fragment which functions as an initiating substrate for enzymatic or synthetic elongation of, for example, a nucleic acid molecule.

The term “promoter” refers to a DNA sequence which directs transcription of DNA to RNA.

A “probe” as used herein is a nucleic acid compound that hybridizes with another nucleic acid compound, and is useful for blot hybridizations, for example. A probe is at least 15 base pairs in length, its sequence being at least 90% identical with the nucleic acid molecules disclosed herein, or fragments thereof, or the complements thereof. A probe may or may not be labeled with a detectable moiety. As used herein, a probe is useful for hybridization analysis to identify sequences homologous to those disclosed herein.

The term “hybridization” as used herein refers to a process in which a single-stranded nucleic acid molecule joins with a complementary strand through nucleotide base pairing. “Selective hybridization” refers to hybridization under conditions of high stringency. The degree of hybridization depends upon, for example, the degree of complementarity, the stringency of hybridization, and the length of hybridizing strands.

The term “stringency” refers to hybridization conditions. High stringency conditions disfavor non-homologous basepairing. Low stringency conditions have the opposite effect. Stringency may be altered, for example, by temperature and salt concentration.

“Low stringency” conditions comprise, for example, a temperature of about 37° C. or less, a formamide concentration of less than about 50%, and a moderate to low salt (SSC) concentration; or, alternatively, a temperature of about 50° C. or less, and a moderate to high salt (SSPE) concentration.

“High stringency” conditions comprise a temperature of about 42° C. or less, a formamide concentration of less than about 20%, and a low salt (SSC) concentration; or, alternatively, a temperature of about 65° C., or less, and a low salt (SSPE) concentration.

“SSC” comprises a hybridization and wash solution. 20×SSC contains 3M sodium chloride, 0.3M sodium citrate, pH 7.0.

“SSPE” comprises a hybridization and wash solution. 1× SSPE contains 180 mM NaCl, 9mM Na₂HPO₄, 0.9 mM NaH₂PO₄ and 1 mM EDTA, pH 7.4.

### DETAILED DESCRIPTION OF THE INVENTION

In yeast and other fungi, for example C. albicans, Aspergillus nidulans, Aspergillus fumigatus, Cryptococcus neofomans, C. Krusei, C. parapsilosis, C. tropicalis, and C. glabrata, IPC synthase catalyzes a step in the synthesis of inositolphosphoryl ceramide from ceramide and phosphatidylinositol (G. Becker and R. Lester, Biosynthesis of phosphoinositol-containing sphingolipid from phosphatidylinositol by a membrane prepartation from Saccharomyces cervisiae. J. Bacteriol. 142, 747-754, 1980). Sphingolipids are necessary for growth and viability of the yeast S. cerevisiae. Since IPC synthase is unique to fungi and plants it is a good target for antifungal therapy in mammals.

The IPC synthase gene of C. glabrata comprises the DNA sequence designated herein as SEQ ID NO 1. The IPC synthase gene of C. Krusei comprises the DNA sequence designated. herein as SEQ ID NO 4. The IPC synthase gene of C. parapsilosis comprises the DNA sequence designated herein as SEQ ID NO 7. The IPC synthase gene of C. tropicalis comprises the DNA sequence designated herein as SEQ ID NO 10. The IPC synthase gene of A. fumigatus is designated herein as SEQ ID NO:13. The IPC synthase gene of A. nidulans is designated herein as SEQ ID NO:16. There are no intervening sequences in these genes. The IPC synthase gene of C. neoformans is designated herein as SEQ ID NO:19 and the cDNA thereof as SEQ ID NO:20. There is one intervening sequence in SEQ ID NO:19, from base pair 1888 through base pair 1939. Those skilled in the art will recognize that owing to the degeneracy of the genetic code (i.e. 64 codons which encode 20 amino acids), numerous “silent” substitutions of nucleotide base pairs could be introduced into these sequences without altering the identity of the encoded amino acid(s) or protein products. All such substitutions are intended to be within the scope of the invention. The genes disclosed and contemplated herein are useful for expressing the protein encoded thereby, in vitro or in a recombinant host cell.

Also contemplated is the use of said genes and fragments thereof as molecular hybridization probes for the identification and isolation of homologous genes.

Also contemplated by the present invention are nucleic acids that hybridize under high stringency conditions to the nucleic acid sequences disclosed herein.

Also contemplated are nucleic acids that are at least 70% identical in sequence to a nucleic acid sequence disclosed herein.

Gene Isolation Procedures

Those skilled in the art will recogize that the IPC synthase genes disclosed herein may be obtained by a plurality of applicable genetic and recombinant DNA techniques including, for example, polymerase chain reaction (PCR) amplification, or de novo DNA synthesis. (See e.g., J. Sambrook et al. Molecular Cloning, 2d Ed. Chap. 14 (1989)).

Skilled artisans will recognize that the IPC synthase genes disclosed herein, or fragments thereof, could be isolated by PCR amplification of genomic DNA isolated from suitable fungal cells using oligonucleotide primers targeted to any suitable region of SEQ ID NO:1, SEQ ID NO:4, SEQ ID NO:7, SEQ ID NO:10, SEQ ID NO:13, SEQ ID NO:16, or SEQ ID NO:19. The skilled artisan understands that the choice of suitable primers may involve routine experimentation to achieve a successful outcome in a PCR amplification and that some trial and error with specific primers may be necessary. Methods for PCR amplification are widely known in the art. See e.g. PCR Protocols: A Guide to Method and Application, Ed. M. Innis et al., Academic Press (1990). The PCR amplification reaction comprises genomic DNA, suitable enzymes, primers, and buffers, and is conveniently carried out in a DNA Thermal Cycler (Perkin Elmer Cetus, Norwalk, Conn.). A positive result is determined by detecting an appropriately-sized DNA fragment following agarose gel electrophoresis.

Protein Production Methods

One of the embodiments of the present invention relates to the purified proteins encoded by the IPC synthase genes disclosed herein, or functionally related proteins.

Skilled artisans will recognize that the proteins of the present invention can be synthesized by a variety of different methods. For example, the amino acid compounds of the invention can be made by chemical methods, well known in the art, including solid phase peptide synthesis or recombinant methods. Both methods are described in U.S. Pat. No. 4,617,149, which hereby is incorporated by reference.

Solid phase chemical synthetic methods for polypeptides are well known in the art, and are described in general texts covering the area. See, e.g., H. Dugas and C. Penney, Bioorganic Chemistry (1981) Springer-Verlag, New York, 54-92. For example, peptides may be synthesized by solid-phase methodology utilizing an Applied Biosystems 430A peptide synthesizer (Applied Biosystems, Foster City, Calif.) and synthesis cycles supplied by Applied Biosystems. Protected amino acids, such as t-butoxycarbonyl-protected amino acids, and other reagents are commercially available from many chemical supply houses.

The proteins of the present invention can also be produced by recombinant DNA methods using a cloned IPC synthase gene described herein. Recombinant methods are preferred if a high yield is desired. Expression of a cloned IPC synthase gene can be carried out in a variety of suitable host cells, well known to those skilled in the art. For this purpose, an IPC synthase gene is introduced into a host cell by any suitable means, well known to those skilled in the art. While chromosomal integration of the cloned IPC synthase gene is within the scope of the present invention, it is preferred that the gene be cloned into a suitable extra-chromosomally maintained expression vector so that the coding region of the IPC synthase gene is operably-linked to a constitutive or inducible promoter.

The basic steps in the recombinant production of the IPC synthase protein are:

a) constructing a natural, synthetic or semi-synthetic DNA encoding IPC synthase;

b) incorporating said DNA into an expression vector in a manner suitable for expressing an IPC synthase protein, either alone or as a fusion protein;

c) transforming an appropriate eucaryotic or prokaryotic host cell with said expression vector,

d) culturing said transformed host cell in a manner to express the IPC synthase protein; and

e) recovering membranes from said host cell and/or purifying the IPC synthase protein by any suitable means, well known to those skilled in the art.

Expressing Recombinant IPC synthase in Procaryotic and Eucaryotic Host Cells

In general, prokaryotes are used for cloning DNA sequences and for constructing the vectors of the present invention. Prokaryotes are also employed in the production of the IPC synthase protein. For example, the Escherichia coli K12 strain 294 (ATCC No. 31446) is particularly useful for expressing heterologous proteins in a procaryotic host. Other strains of E. coli, bacilli such as Bacillus subtilis, enterobacteriaceae such as Salmonella typhimurium or Serratia marcescans, various Pseudomonas species and other bacteria, such as Streptomyces, may also be employed as host cells in cloning and expressing the recombinant proteins of this invention.

Promoters that are suitable for driving expression of genes in prokaryotes include b-lactamase [e.g. vector pGX2907, ATCC 39344, contains a replicon and b-lactamase gene], lactose systems [Chang et al., Nature_(London), 275:615 (1978); Goeddel et al., Nature (London), 281:544 (1979)], alkaline phosphatase, and the tryptophan (trp) promoter system [vector pATH1 (ATCC 37695) which is designed to facilitate expression of an open reading frame as a trpE fusion protein under the control of the trp promoter]. Hybrid promoters such as the tac promoter (isolatable from plasmid pDR540, ATCC-37282) are also suitable. Still other bacterial promoters, whose nucleotide sequences are generally known, enable one of skill in the art to ligate such promoter sequences to DNA encoding the proteins of the instant invention using linkers or adapters to supply any required restriction sites. Promoters for use in bacterial systems also will contain a Shine-Dalgarno sequence operably-linked to the DNA encoding the desired polypeptides. These examples are illustrative rather than limiting.

The proteins of this invention may be synthesized either by direct expression or as a fusion protein comprising the protein of interest as a translational fusion with another protein or peptide, which may be removable by enzymatic or chemical cleavage. It is often observed in the production of certain peptides in recombinant systems that expression as a fusion protein prolongs the lifespan, increases the yield of the desired peptide, or provides a convenient means of purifying the protein. A variety of peptidases (e.g. enterokinase and thrombin) which cleave a polypeptide at specific sites or digest the peptides from the amino or carboxy termini (e.g. diaminopeptidase) of the peptide chain are known. Furthermore, particular chemicals (e.g. cyanogen bromide) will cleave a polypeptide chain at specific sites. The skilled artisan will appreciate the modifications necessary to the amino acid sequence (and synthetic or semi-synthetic coding sequence if recombinant means are employed) to incorporate site-specific internal cleavage sites. See e.g., P. Carter, “Site Specific Proteolysis of Fusion Proteins”, Chapter 13, in Protein Purification: From Molecular Mechanisms to Large Scale Processes, American Chemical Society, Washington, D.C. (1990).

In addition to prokaryotes, mammalian host cells and eucaryotic microbes, such as yeast, may also be used to express the proteins of this invention. The simple eucaryote Saccharomyces cerevisiae is the most commonly used eucaryotic microorganism, although a number of other yeasts, such as Kluyveromyces lactis, are also suitable. For expression in Saccharomyces, the plasmid YRp7 (ATCC-40053), for example, may be used. See, e.g., D. Stinchcomb et al., Nature, 282:39 (1979); J. Kingsman et al., Gene, 7:141 (1979); S. Tschemper et al., Gene, 10:157 (1980). Plasmid YRp7 contains the TRP1 gene which provides a selectable marker for use in a trp1 auxotrophic mutant.

Purification of Recombinantly-Produced IPC Synthase

An expression vector carrying a cloned IPC synthase gene or cDNA from any of the fungi disclosed herein (SEQ ID NO:1, SEQ ID NO:4, SEQ ID NO:7, SEQ ID NO:10, SEQ ID NO:13, SEQ ID NO:16, SEQ ID NO:19, or SEQ ID NO:20) is transformed or transfected into a suitable host cell using standard methods. Cells which contain the vector are propagated under conditions suitable for expression of the IPC synthase protein. If an IPC synthase gene is under the control of an inducible promoter then growth conditions would incorporate the appropriate inducer. The recombinantly-produced IPC synthase protein may be purified from cellular extracts of transformed cells by any suitable means. In a preferred method for protein purification, an IPC synthase gene used in transforming a host cell is modified at the 5′ end to incorporate several histidine residues at the amino terminus of the encoded IPC synthase protein. This “histidine tag” enables a single-step protein purification method referred to as “immobilized metal ion affinity chromatography” (IMAC), essentially as described in U.S. Pat. No. 4,569,794, which hereby is incorporated by reference. The IMAC method enables rapid isolation of substantially pure protein.

IPC synthase activity can be detected in membranes from recombinant cells transformed with the genes disclosed herein, or in the membranes of non-transformed fungal cells that express said genes. Said membranes are a useful source of IPC synthase activity and can be used as a reagent in an assay for IPC synthase activity.

Other embodiments of the present invention relate to isolated nucleic acid sequences which encode SEQ ID NO:2, SEQ ID NO:5, SEQ ID NO:8, SEQ ID NO:11, SEQ ID NO:14, SEQ ID NO:17, or SEQ ID NO:21 or fragments thereof. As skilled artisans will recognize, the amino acid compounds of the invention can be encoded by a multitude of different nucleic acid sequences due to the degeneracy of the genetic code. Because these alternative nucleic acid sequences would encode the same amino acid sequences, the present invention further comprises these alternate nucleic acid sequences.

The IPC synthase genes comprising the present invention may be produced using synthetic methods well known in the art. See, e.g., E. L. Brown, R. Belagaje, M. J. Ryan, and H. G. Khorana, Methods in Enzymology, 68:109-151 (1979). The DNA segments corresponding to an IPC synthase gene could be generated using a conventional DNA synthesizing apparatus, such as the Applied Biosystems Model 380A or 380B DNA synthesizers (Applied Biosystems, Inc., 850 Lincoln Center Drive, Foster City, Calif. 94404) which employ phosphoramidite chemistry. Alternatively, phosphotriester chemistry may be employed to synthesize the nucleic acids of this invention. [See, e.g., M. J. Gait, ed., Oligonucleotide Synthesis, A Practical Approach, (1984).]

In an alternative method, IPC synthase DNA sequences comprising a portion or all of SEQ ID NO:1, SEQ ID NO:4, SEQ ID NO:7, SEQ ID NO:10, SEQ ID NO:13, SEQ ID NO:16, SEQ ID NO:19, or SEQ ID NO:20 can be generated from fungal genomic DNA using suitable oligonucleotide primers complementary to these sequences or region therein, utilizing the polymerase chain reaction as described in U.S. Pat. No. 4,889,818, which is incorporated herein by reference. Protocols for performing the PCR are disclosed in, PCR Protocols: A Guide to Method and Applications, Ed. Michael A. Innis et al., Academic Press, Inc. (1990), which hereby is incorporated by reference.

The ribonucleic acids of the present invention may be prepared using polynucleotide synthetic methods discussed supra, or they may be prepared enzymatically using RNA polymerases to transcribe a DNA template.

The most preferred system for preparing the ribonucleic acids of the present invention employs the RNA polymerase from the bacteriophage T7 or bacteriophage SP6. These RNA polymerases are highly specific and require the insertion of bacteriophage-specific sequences at the 5′ end of the template to be transcribed. See, J. Sambrook, et al., supra, at 18.82-18.84.

This invention also provides nucleic acids, RNA or DNA, which are complementary to SEQ ID NO:1, SEQ ID NO:3, SEQ ID NO:4, SEQ ID NO:6, SEQ ID NO:7, SEQ ID NO:9, SEQ ID NO:10, SEQ ID NO:12, SEQ ID NO:13, SEQ ID NO:15, SEQ ID NO:16, SEQ ID NO:18, SEQ ID NO:19, SEQ ID NO:20, or SEQ ID NO:22.

The present invention also provides probes and primers useful for molecular biology techniques. A compound that is SEQ ID NO:1, SEQ ID NO:3, SEQ ID NO:4, SEQ ID NO:6, SEQ ID NO:7, SEQ ID NO:9, SEQ ID NO:10, SEQ ID NO:12, SEQ ID NO:19, SEQ ID NO:20, or SEQ ID NO:22, or a complementary sequence thereof, or a fragment thereof, and which is at least 15 base pairs in length, and which will selectively hybridize to C. glabrata, C. krusei, C. parapsilosis, C. tropicalis or C. neoformans DNA or mRNA encoding IPC synthase, is provided. Preferably, the compound is DNA.

The probes and primers contemplated herein can be prepared enzymatically by well known methods (See e.g. Sambrook et al. supra).

Another aspect of the present invention relates to recombinant DNA cloning vectors and expression vectors comprising the nucleic acids described and contemplated herein. Many of the vectors encompassed within this invention are described above. The preferred nucleic acid vectors are those which comprise DNA. The most preferred recombinant DNA vectors comprise the isolated DNA sequences SEQ ID NO:1, SEQ ID NO:4, SEQ ID NO:7, SEQ ID NO:10, SEQ ID NO:13, SEQ ID NO:16, SEQ ID NO:19, or SEQ ID NO:20.

The skilled artisan understands that choosing the most appropriate cloning vector or expression vector depends upon a number of factors including the availability of appropriate restriction enzyme sites, the type of host cell into which the vector is to be transfected or transformed, the purpose of the transfection or transformation (e.g., stable transformation as an extrachromosomal element, or integration into the host chromosome), the presence or absence of readily assayable or selectable markers (e.g., antibiotic resistance markers, metabolic markers, or the like), and the number of copies of the gene to be present in the host cell.

Vectors suitable to carry the nucleic acids of the present invention include RNA viruses, DNA viruses, lytic bacteriophages, lysogenic bacteriophages, stable bacteriophages, plasmids, viroids, and the like. The most preferred vectors are plasmids.

When preparing an expression vector the skilled artisan understands that there are many variables to be considered, for example, whether to use a constitutive or inducible promoter. Inducible promoters are preferred because they may be the basis for high level and regulatable expression of an operably-linked gene. The skilled artisan will recognize a number of inducible promoters and inducers, for example, carbon source, metal ions, heat, and others known to the skilled artisan. The practitioner also understands that the amount of nucleic acid or protein to be produced dictates, in part, the selection of the expression system. The addition of certain nucleotide sequences, such as a sequence encoding a signal peptide preceding the coding sequence, is useful to direct localization of the resulting polypeptide.

Host cells harboring the nucleic acids disclosed herein are also provided by the present invention. A preferred host is E. coli, into which has been transfected or transformed a vector that comprises a nucleic acid of the present invention.

The present invention also provides a method for constructing a recombinant host cell capable of expressing SEQ ID NO:2, SEQ ID NO:5, SEQ ID NO:8, SEQ ID NO:11, SEQ ID NO:14, SEQ ID NO:17, or SEQ ID NO:21 said method comprising transforming a host cell with a recombinant DNA vector that comprises an isolated DNA sequence that encodes one of these sequences. Suitable host cells include any strain of E. coli or fungal cell that can accomodate high level expression of a gene(s) introduced by transformation or transfection. Preferred vectors for expression are those that comprise SEQ ID NO:1, SEQ ID NO:4, SEQ ID NO:7, SEQ ID NO:10, SEQ ID NO:13, SEQ ID NO:16, SEQ ID NO:19, or SEQ ID NO:20. Transformed host cells may be cultured under conditions well known to skilled artisans such that SEQ ID NO:2, SEQ ID NO:5, SEQ ID NO:8, SEQ ID NO:11, SEQ ID NC):14, SEQ ID NO:17, or SEQ ID NO:21 is expressed, thereby producing fungal IPC synthase protein in a recombinant host cell.

For the purpose of identifying or developing antifungal compounds, it would be desirable to determine those agents which inhibit IPC synthase activity. A method for determining whether a substance will inhibit the enzymatic reaction catalyzed by IPC synthase comprises contacting a source of IPC synthase activity (e.g. membrane preparation from a cell that expresses IPC synthase), or a purified IPC synthase protein, or fragment exhibiting said synthase activity, with a test substance and monitoring IPC synthase activity by any suitable means.

The instant invention provides such a screening system useful for discovering agents which inhibit an IPC synthase, said screening system comprising the steps of:

a) preparing a source of IPC synthase, or IPC synthase protein, or subunit thereof;

b) exposing said IPC synthase or source thereof to a test inhibitor;

c) introducing substrate; and

d) quantifying the loss of activity of said IPC synthase.

Utilization of the screening system described above provides a means to determine compounds that may interfere with fungal sphingolipid biosynthesis. This screening system may be adapted to automated procedures such as a PANDEX® (Baxter-Dade Diagnostics) system allowing for efficient high-volume screening of potential therapeutic agents.

In such a screening protocol IPC synthase or subunit thereof is prepared as described herein, preferably using recombinant DNA technology. Alternatively, the reaction can be carried out using membranes from cells that express ICP synthase, as a source of IPC synthase activity. Preferably, the cells are recombinant cells that incorporate a recombinantly-expressed IPC synthase into the cell membrane. Most preferably, the recombinant cells are yeast cells. A sample of a test compound is then introduced into a reaction vessel containing IPC synthase activity, followed by the addition of enzyme substrate. Alternatively, substrate may be added simultaneously with the test compound.

The following examples more fully describe the present invention. Those skilled in the art will recognize that the particular reagents, equipment, and procedures described are merely illustrative and are not intended to limit the present invention in any manner.

### EXAMPLE 1

### Construction of a DNA Vector for Expressing the C. glabrata IPC synthase Gene in Homologous or Heterologous Host

An expression vector suitable for expressing the IPC synthase gene of C. glabrata (SEQ ID NO:1) in E. coli contains an origin of replication (Ori), an ampicillin resistance gene (Amp) useful for selecting cells that have incorporated the vector following tranformation. The vector also includes the T7 promoter and T7 terminator sequences in operable linkage to the coding region of the IPC synthase gene. Parent plasmid pET11A (obtained from Novogen, Madison, Wis.) is linearized by digestion with appropriate endonucleases and ligated to a DNA fragment comprising the coding region of the C. glabrata IPC synthase gene.

The IPC synthase gene ligated into the expression vector is modified at the 5′ end (amino terminus of encoded protein) in order to simplify purification of the encoded IPC synthase protein product. For this purpose, an oligonucleotide encoding 8 histidine residues and a factor Xa cleavage site is inserted after the ATG start codon at nucleotide positions 1 to 3 of SEQ ID NO:1. Placement of the histidine residues at the amino terminus of the encoded protein enables the IMAC one-step protein purification procedure (See below).

### EXAMPLE 2

### Expression of C. glabrata IPC synthase Gene in Echerichia coli and Purification of IPC Synthase Enzyme

A plasmid from Example 1 is transformed into E. coli BL21 (DE3)(hsdS gal lcIts857 ind1Sam7nin5lacUVS-T7gene 1) using standard methods (See e.g. Sambrook et al. Supra). Transformants, selected for resistance to ampicillin, are chosen at random and tested for the presence of the vector by agarose gel electrophoresis using quick plasmid preparations. Colonies that contain the vector are grown, processed, and the protein encoded by the IPC synthase gene is purified by immobilized metal ion affinity chromatography (IMAC), essentially as described in U.S. Pat. No. 4,569,794, the entire contents of which is hereby incorporated by reference.

Briefly, the IMAC column is prepared as follows. A metal-free chelating resin (e.g. SEPHAROSE 6B IDA, Pharmacia) is washed in distilled water to remove preservative substances and infused with a suitable metal ion [e.g. Ni(II), Co(II), or Cu(II)] by adding a 50 mM metal chloride or metal sulfate aqueous solution until about 75% of the interstitial spaces of the resin is saturated with colored metal ion. The column is then ready to receive a crude cellular extract containing the IPC synthase protein product encoded by the vector.

After removing unbound proteins and other materials by washing the column with suitable buffer, pH 7.5, the bound protein is eluted in buffer at pH 4.3 essentially as described in U.S. Pat. No. 4,569,794.

### EXAMPLE 3

### Biochemical Assay for Inhibitors of IPC Synthase Using Fungal Membrane Preparations

The activity of the IPC synthase enzyme is assayed by preparing membranes from C. glabrata, for example, and using said membranes as a source of IPC synthase activity.

A suitable, rich medium, for example YEPD, is innoculated with a culture and allowed to grow overnight at room temperature with vigorous shaking. About 250 ml of fresh medium containing 20 ug/ml myo-inositol is innoculated with the overnight culture, and grown overnight at 30° C. Cells are harvested by centrifugation and resuspended in ice cold 50 mM potassium phosphate buffer, pH 7. Cells are washed twice in the same buffer and then resuspended in the same buffer containing 5 mM dithiothreitol (DTT), 1 ug,/ml aprotinin, 0.6 uM leupeptin, 1 mM PMSF, and 1 ug/ml pepstatin A. Cells are ruptured using glass beads in a procedure that involves 5 successive vortexings each for 30 seconds followed by 2 to 5 minute intervals of rest on ice. Membranes are pelleted by centrifugation at 100,000×g for 1 hour at 4° C. The pellet is resuspended in cold buffer containing DTT and protease inhibitors and disrupted further by Dounce homogenization with 5 to 6 strokes on ice. Membranes are mixed with glycerol to a final concentration of 33% and stored frozen at −80° C. For use as “protein stock,” thaw and dilute to 4.8 mg/ml in 0.05 M potassium phosphate (Kpi) buffer, pH 7.0 on ice.

IPC synthase is assayed by any suitable method. For example:

Final Assay Conditions:

5-50 uM NBD-C6-ceramide (from Molecular Probes)

1 mM phosphatidylinositol (Sigma-soybean)

2 mM CHAPS

50 mM potassium phosphate buffer, pH 7.0

Up to 2.5% organic co-solvent

Protein to 1.2 mg/ml

Final Vol. 100 ul

2X Master Mix (for 15 samples)

46 ul of 1 mg/ml NBD-C6-ceramide in MeOH

142 ul of 10 mg/ml phosphatidylinositol in CHCl₃

Dry under vacuum

Redissolve by adding in order

160 ul 20 mM CHAPS

560 ul deionized water

80 ul 0.5 M Kpi buffer, pH 7.0

Sonicate 5 minutes.

Aliquot 50 ul of 2X master mix to screw cap microfuge at room temperature. Add 20 ul of 0.05 M Kpi buffer pH 7.0 to each sample followed by 5 ul of test compound or extract. The reaction is initiated by adding 25 ul of protein stock and incubating for 1 hour in a 30° C. water bath. Reactions are terminated by adding 900 ul of cold absolute methanol. Samples are stored at −20° C. for 1 hour and centrifuged at 14000×g at 4° C. for 30 minutes.

The reaction is monitored by HPLC on a Beckman Ultrasphere-ODS 5 um, 150×4 mm column. The chromatogram is developed in 87% MeOH/13% 50 mM TEAP, pH 5.8, at a flow rate of 1 ml/min at room temperature through a Beckman Model 157 Fluorescence detector, using NBD-C₆-phosphatidylcholine as an internal standard.

Inhibition studies are carried out using the same reaction conditions described in the preceding paragraph. Compounds to be studied for inhibitory activity are added to final concentrations of between 1 pM and 10 mM.

### EXAMPLE 4

### Expression of C. parapsilosis (SEQ ID NO:7) IPC Synthase Gene in S. cerevisiae

A yeast/E. coli shuttle vector suitable for expressing the C. parapsilosis IPC synthase gene (SEQ ID NO:7) in S. cerevisiae is constructed in parent plasmid YEp351 (See J. Hill et. al., Yeast, 2, 163-167, 1986), which contains the multiple cloning region of pUC18, the Amp^{R} gene for selection in E. coli, 2μ replicon, and the LEU2 gene for selection in yeast. A fragment containing the C. parapsilosis IPC synthase gene is prepared by PCR amplification. Suitable primers to the 5′ and 3′ ends of the coding region disclosed in SEQ ID NO:7 are constructed to contain BamH1 cloning sites in addition to IPC synthase coding information. After PCR amplification of C. parapsilosis genomic DNA, the amplified fragment is purified by any sutiable method, for example, gel purification of an appropriately sized fragment, followed by treatment with restriction enzyme BamH1, and ligation to BamH1 digested YEp351. The recombinant plasmid carrying the IPC synthase gene is transformed into any suitable leu strain of S. cerevisiae and transformants selected for growth in a medium that lacks added leucine. Membranes from tranformants are prepared, and IPC synthase activity assayed as in Example 3.

We claim:

1. A substantially pure IPC synthase protein from a fungal cell other than Saccharomyces cerevisiae.

2. A substantially pure IPC synthase protein from a fungal cell comprising an amino acid sequence selected from the group consisting of:
a) SEQ ID NO:2;
b) SEQ ID NO:5;
c) SEQ ID NO:8;
d) SEQ ID NO:11; or
e) SEQ ID NO:21.

3. An isolated nucleic acid encoding a protein of claim 1.

4. An isolated nucleic acid encoding a protein of claim 2.

5. An isolated nucleic acid compound encoding a fungal IPC synthase protein of claim 2, or fragment thereof, wherein said compound has a sequence selected from the group consisting of:
(a) SEQ ID NO:1;
(b) SEQ ID NO:3;
(c) SEQ ID NO:4;
(d) SEQ ID NO:6;
(e) SEQ ID NO:7;
(f) SEQ ID NO:9;
(g) SEQ ID NO:10;
(h) SEQ ID NO:12;
(i) SEQ ID NO:19;
(j) SEQ ID NO:20;
(k) SEQ ID NO:22;
(l) a nucleic acid compound complementary to (a), (b), (c), (d), (e), (f), (g), (h), (i), (j), or (k); and
(m) a fragment of (a), (b), (c), (d), (e), (f), (g), (h.), (i), (j), (k), or (l) that is at least 18 base pairs in length and which will selectively hybridize to IPC synthase genomic DNA.

6. An isolated nucleic acid compound of claim 5 wherein the sequence of said compound is SEQ ID NO:1, SEQ ID NO:4, SEQ ID NO:7, SEQ ID NO:10, SEQ ID NO:19, or SEQ ID NO:20, or a sequence complementary thereof.

7. An isolated nucleic acid compound of claim 5 wherein the sequence of said compound is SEQ ID NO:3, SEQ ID NO:5, SEQ ID NO:8, SEQ ID NO:11, or SEQ ID NO:22, or a sequence complementary thereof.

8. A vector comprising an isolated nucleic acid compound of claim 5.

9. A vector, as in claim 8, wherein said isolated nucleic acid compound is SEQ ID NO:1, SEQ ID NO:4, SEQ ID NO:7, SEQ ID NO:10, SEQ ID NO:19, or SEQ ID NO:20, operably-linked to a promoter sequence.

10. A host cell containing a vector of claim 8.

11. A host cell containing a vector of claim 9.

12. A method for constructing a recombinant host cell having the potential to express SEQ ID NO:2, SEQ ID NO:5, SEQ ID NO:8, SEQ ID NO:11, or SEQ ID NO:21, said method comprising introducing into said host cell by any suitable means a vector of claim 9.

13. A method for expressing SEQ ID NO:2, SEQ ID NO:5, SEQ ID NO:8, SEQ ID NO:11, or SEQ ID NO:21 in the recombinant host cell of claim 10, said method comprising culturing said recombinant host cell under conditions suitable for gene expression.

14. A method for identifying inhibitory compounds of fungal IPC synthase protein activity, comprising the steps of:
a) admixing in a suitable reaction buffer
i) a source of IPC synthase protein;
ii) a suitable substrate;
iii) a test inhibitory compound;
b) measuring by any suitable means an amount of product formed; and
c) comparing the amount of product formed at step (b) with a control reaction, said control reaction comprising steps (a) (i), (a) (ii), and (b) and wherein said control reaction lacks said test inhibitory compound.

15. A method, as in claim 14, wherein said synthase protein is selected from the group consisting of SEQ ID NO:2, SEQ ID NO:5, SEQ ID NO:8, SEQ ID NO:11, SEQ ID NO:14, SEQ ID NO:17, and SEQ ID NO:21.

16. A method, as in claim 14, wherein at step (a)(i) said source comprises membranes from a cell that expresses IPC synthase.

17. A method, as in claim 16, wherein said cell is a recombinant host cell that expresses a vector-borne IPC synthase gene selected from the group consisting of SEQ ID NO:1, SEQ ID NO:4, SEQ ID NO:7, SEQ ID NO:10, SEQ ID NO:13, SEQ ID NO:16, SEQ ID NO:19, and SEQ ID NO:20.

18. An isolated nucleic acid compound for use as a probe or primer consisting of a nucleic acid molecule wherein the sequence of said nucleic acid is identical with a fragment of or the entirety of SEQ ID NO:1, SEQ ID NO:4, SEQ ID NO:7, SEQ ID NO:10, or SEQ ID NO:19, or to the complement thereof.

19. An isolated nucleic acid compound for use as a probe or primer consisting of a nucleic acid molecule that is at least 20 base pairs in length, and wherein the sequence of said nucleic acid is at least 90% identical with a fragment of, or the entirety of, SEQ ID NO:1, SEQ ID NO:4, SEQ ID NO:7, SEQ ID NO:10, or SEQ ID NO:19, or to the complement thereof.

20. An isolated nucleic acid compound as in claim 19 wherein said nucleic acid hybridizes to said SEQ ID NO:1, SEQ ID NO:4, SEQ ID NO:7, SEQ ID NO:10, or SEQ ID NO:19, or to the complement thereof.


SEQUENCE LISTING (24 sequences)

SEQ ID NO: 1 - DNA, 2000 bp, Candida glabrata
  CDS (501)..(1748)
aaaacgtcattttaaaattaggctttagattgtaactcagatgattttcgtcaatccagtgtacgaaacttggcaaatgctaaaacttcaacaaataaatcatcatccctaatactagtgtaaataatttaatgtacatggtgttctatttctaatgtagaaatatgtatatgacattagatcaactcatattgttcaaaactctgtcaaaataatttagtgaggcaaaggtgtcgtcccccgaatgaacttgtaactcaccttgaaggagatatttgcatttacttgataatccgaaacttttgcaacttaacattttaaaactagaagttatttggtatctggtattaatttttattagctttgctgattacgacacgttacgaaggaagatattgacgctcagaatttttatttaggagaaatttatcataatagacaatatttgccattaccgatttctaactaaatatcataatcatagttattagacaaattcaatgagacttggaagatggtttgctattttgagctcaaaattatgctcagatacagatgtaactaagttggagagaagtcttgatgtaagggtcacattaagacgactcaggagttacagacccacaataggtgacacagttcattattcatttttgtcaatgattctattgtattcatattttgctaatccactaccattcattgtgaaaacattgatagcgatgattttaatgactctgtttgtcgtcccgatgacatctcaatttttctttaatgcacttccaatattgacatggctaatcttgtattttacatcatcacatattcctaatagtcatagaccttcaatttctgttgaggtgcttcctgccattgaaactatcctgtatggcgataacttaagtgagattttggctgcatggcaaaatacaattctagatatcctagcttggattccatatggtctgtttcactttggcgcaccatttgtggttgcaattgttttatttatatttggaccaccgacagttcttcagggttatgcgtttgcgtttggttatgtgaatttatttggggtgttattccaaaacatatttcccgccgcggctccttggtataaaatacattatggactccaagttgcaaattacagtatcaaaggtatgcctggtggactttctagaattgatgatcttttgggtgtgaatttatattcaagtggttttaagaactctgctgtcgtttttggagcttttccttctcttcattctgcatgtgcaactatggaagcactcttcttctcatattgttttccaagcttgacaccgctctttattttctacgtttggtggttatggtggtctacaatgtatcttactcatcattattttgttgatctaacagcaggttctgttttggcttatttcatatttcaatataccaaatacacacatctacctattgttgatccaaatctgctcttccgttggtcctacgacagtattgaattttacgatgttaagaacaatgaccctttatccattacaaataacgatatagagagtatacctctaaacaccattgaactagatacagaagaagtgttcaatattgagcgggaaaaatctaggacaccagaattgtcacatgcagtgtcttcatcttctctagctaacggtcgctctcaaacatatccaaatactggaaatacattcaaatccaagacaagactgagtaaactggtcatttaaaaccagtatttataaatagagactatcttcttctactgtacatacaatgatctttctatattctaacgaaatcaataagtgcatactatattcatttaacatgatcaatattatttaacagatccattaggtgctggcttcccaagatagttaccaaaacgataattttttctcaaaatttgccctttgagaaaaacctccgtctagtcaccccttaccacacagcacaaactgaaaatcgatgggata

SEQ ID NO: 2 - protein, 416 aa, Candida glabrata
MRLGRWFAILSSKLCSDTDVTKLERSLDVRVTLRRLRSYRPTIGDTVHYSFLSMILLYSYFANPLPFIVKTLIAMILMTLFVVPMTSQFFFNALPILTWLILYFTSSHIPNSHRPSISVEVLPAIETILYGDNLSEILAAWQNTILDILAWIPYGLFHFGAPFVVAIVLFIFGPPTVLQGYAFAFGYVNLFGVLFQNIFPAAAPWYKIHYGLQVANYSIKGMPGGLSRIDDLLGVNLYSSGFKNSAVVFGAFPSLHSACATMEALFFSYCFPSLTPLFIFYVWWLWWSTMYLTHHYFVDLTAGSVLAYFIFQYTKYTHLPIVDPNLLFRWSYDSIEFYDVKNNDPLSITNNDIESIPLNTIELDTEEVFNIEREKSRTPELSHAVSSSSLANGRSQTYPNTGNTFKSKTRLSKLVI

SEQ ID NO: 3 - RNA, 1248 nt, Candida glabrata
augagacuuggaagaugguuugcuauuuugagcucaaaauuaugcucagauacagauguaacuaaguuggagagaagucuugauguaagggucacauuaagacgacucaggaguuacagacccacaauaggugacacaguucauuauucauuuuugucaaugauucuauuguauucauauuuugcuaauccacuaccauucauugugaaaacauugauagcgaugauuuuaaugacucuguuugucgucccgaugacaucucaauuuuucuuuaaugcacuuccaauauugacauggcuaaucuuguauuuuacaucaucacauauuccuaauagucauagaccuucaauuucuguugaggugcuuccugccauugaaacuauccuguauggcgauaacuuaagugagauuuuggcugcauggcaaaauacaauucuagauauccuagcuuggauuccauauggucuguuucacuuuggcgcaccauuugugguugcaauuguuuuauuuauauuuggaccaccgacaguucuucaggguuaugcguuugcguuugguuaugugaauuuauuugggguguuauuccaaaacauauuucccgccgcggcuccuugguauaaaauacauuauggacuccaaguugcaaauuacaguaucaaagguaugccugguggacuuucuagaauugaugaucuuuugggugugaauuuauauucaagugguuuuaagaacucugcugucguuuuuggagcuuuuccuucucuucauucugcaugugcaacuauggaagcacucuucuucucauauuguuuuccaagcuugacaccgcucuuuauuuucuacguuuggugguuaugguggucuacaauguaucuuacucaucauuauuuuguugaucuaacagcagguucuguuuuggcuuauuucauauuucaauauaccaaauacacacaucuaccuauuguugauccaaaucugcucuuccguugguccuacgacaguauugaauuuuacgauguuaagaacaaugacccuuuauccauuacaaauaacgauauagagaguauaccucuaaacaccauugaacuagauacagaagaaguguucaauauugagcgggaaaaaucuaggacaccagaauugucacaugcagugucuucaucuucucuagcuaacggucgcucucaaacauauccaaauacuggaaauacauucaaauccaagacaagacugaguaaacuggucauu

SEQ ID NO: 4 - DNA, 2000 bp, Candida kruisii
  CDS (300)..(1739)
acagtaaaaagaaattgcgctgcatgaaacttttttttttttttttttcggtgtttcttctcgtttttctcgtttttctcgtttttctcggtttcttttgagaatgtaatgaaatttggaaactgtacaaacattctcagtgttgatattaacaataataaccacattgacattgacaacatcattattaggttctttccatttgtattatttccatctcggacacgtcagctatagaccgtcgacactccctattccctgcaccaactaggaagaacaggaagaacaagaagaacagaatgctctcgctcccggcccttattaggcgcctgtattcctcctttatttcggcaagagaaaagggtaattcattgcctgcattaaagacatcctttaaccccctccctaacttcaagaccttcccggagagactggtctcggcatccatatccaatgtggtccatttcacgattatctttcttgtctttgtcttctccatctcaataatgcctgtacccctacttgtgaaagccattcttatcattgtcttgatttacatcaactttgctccaattttcgcccagttctttaacggcggcttgccgatcctctcatgggtcattttgttctttaactcaaagaacatacctctgtcaatgaagagacccatctccgtcaaggtcttgcccgcattggagacaatcttctatggcgataatttatctgaaatcttggcttctcatacaaacactttcttggacttgtttgcttggttcccatatggtatagtccatttctccctgccctttgttgtggcagcccttatttttttatttgccccccctaaaaccttaccccaattttgttggtcctttggttatatgaatttggttggcgtcattatccaaaacttggttttctcatgtgctccaccatggtataaagtgctgcatgggttggacaaggcaaactattcaatgaagggatcgccaggaggattgggtagaatcgatggactgtttggtttcgatatgtacacctcgggtttcaccaattctcccttgattttcggtgcaatgccctccttgcattcgggatgttcaactatggacgcattatggttgtcttatctattccctaaattcacccctctatttgttttctacgtttgctggttatggttctctacaatgtatttgactcatcattacttcatcgatgttgttgttggttcttgtttggcaattggattcttcactgcagtcaaatggagaggcagattacccgtaaatgatctattttgtaggtgggaccatgataccctgaaattccatgacatttggaacgaggacccactgagggagaactatcccttggacttggagtcttcaaaagacttggaaaatcagatacctttgagtgatttgaattcatcatgtgatgaacaaccaagttcaccaaagggatttaataggaaccagaataataacttgggtgataagggacagggattagaacaagtagacgatcaaggagaagaatatgacaattacgattttgatgattctgatgacgaccaaattgctgaagctaatgatgtggagaaatcggctaacaaaacaataaagaagactccaaagtcatacaaggaatcattcgaagcaacattatctacaaacttgaactcacaaattacaaatttcccatcttccactcatatacctcttaattccagtcgttgacacaaactcggttctttaataactaccacttcgccctttacttagcgtctttgaaattatagatctggaattagaaataaactacctttttttttggccaattggaaaaatttaatacaactcttatcaagtcactcctttattttcaatctttttctttatgtacatgtaattttacagagttcaatagttaattatttaattacttattttcactttatagttacactcttctccccacgtccttttttcttatca

SEQ ID NO: 5 - protein, 480 aa, Candida kruisii
MLSLPALIRRLYSSFISAREKGNSLPALKTSFNPLPNFKTFPERLVSASISNVVHFTIIFLVFVFSISIMPVPLLVKAILIIVLIYINFAPIFAQFFNGGLPILSWVILFFNSKNIPLSMKRPISVKVLPALETIFYGDNLSEILASHTNTFLDLFAWFPYGIVHFSLPFVVAALIFLFAPPKTLPQFCWSFGYMNLVGVIIQNLVFSCAPPWYKVLHGLDKANYSMKGSPGGLGRIDGLFGFDMYTSGFTNSPLIFGAMPSLHSGCSTMDALWLSYLFPKFTPLFVFYVCWLWFSTMYLTHHYFIDVVVGSCLAIGFFTAVKWRGRLPVNDLFCRWDHDTLKFHDIWNEDPLRENYPLDLESSKDLENQIPLSDLNSSCDEQPSSPKGFNRNQNNNLGDKGQGLEQVDDQGEEYDNYDFDDSDDDQIAEANDVEKSANKTIKKTPKSYKESFEATLSTNLNSQITNFPSSTHIPLNSSR

SEQ ID NO: 6 - RNA, 2000 nt, Candida kruisii
acaguaaaaagaaauugcgcugcaugaaacuuuuuuuuuuuuuuuuuucgguguuucuucucguuuuucucguuuuucucguuuuucucgguuucuuuugagaauguaaugaaauuuggaaacuguacaaacauucucaguguugauauuaacaauaauaaccacauugacauugacaacaucauuauuagguucuuuccauuuguauuauuuccaucucggacacgucagcuauagaccgucgacacucccuauucccugcaccaacuaggaagaacaggaagaacaagaagaacagaaugcucucgcucccggcccuuauuaggcgccuguauuccuccuuuauuucggcaagagaaaaggguaauucauugccugcauuaaagacauccuuuaacccccucccuaacuucaagaccuucccggagagacuggucucggcauccauauccaaugugguccauuucacgauuaucuuucuugucuuugucuucuccaucucaauaaugccuguaccccuacuugugaaagccauucuuaucauugucuugauuuacaucaacuuugcuccaauuuucgcccaguucuuuaacggcggcuugccgauccucucaugggucauuuuguucuuuaacucaaagaacauaccucugucaaugaagagacccaucuccgucaaggucuugcccgcauuggagacaaucuucuauggcgauaauuuaucugaaaucuuggcuucucauacaaacacuuucuuggacuuguuugcuugguucccauaugguauaguccauuucucccugcccuuuguuguggcagcccuuauuuuuuuauuugcccccccuaaaaccuuaccccaauuuuguugguccuuugguuauaugaauuugguuggcgucauuauccaaaacuugguuuucucaugugcuccaccaugguauaaagugcugcauggguuggacaaggcaaacuauucaaugaagggaucgccaggaggauuggguagaaucgauggacuguuugguuucgauauguacaccucggguuucaccaauucucccuugauuuucggugcaaugcccuccuugcauucgggauguucaacuauggacgcauuaugguugucuuaucuauucccuaaauucaccccucuauuuguuuucuacguuugcugguuaugguucucuacaauguauuugacucaucauuacuucaucgauguuguuguugguucuuguuuggcaauuggauucuucacugcagucaaauggagaggcagauuacccguaaaugaucuauuuuguaggugggaccaugauacccugaaauuccaugacauuuggaacgaggacccacugagggagaacuaucccuuggacuuggagucuucaaaagacuuggaaaaucagauaccuuugagugauuugaauucaucaugugaugaacaaccaaguucaccaaagggauuuaauaggaaccagaauaauaacuugggugauaagggacagggauuagaacaaguagacgaucaaggagaagaauaugacaauuacgauuuugaugauucugaugacgaccaaauugcugaagcuaaugauguggagaaaucggcuaacaaaacaauaaagaagacuccaaagucauacaaggaaucauucgaagcaacauuaucuacaaacuugaacucacaaauuacaaauuucccaucuuccacucauauaccucuuaauuccagucguugacacaaacucgguucuuuaauaacuaccacuucgcccuuuacuuagcgucuuugaaauuauagaucuggaauuagaaauaaacuaccuuuuuuuuuggccaauuggaaaaauuuaauacaacucuuaucaagucacuccuuuauuuucaaucuuuuucuuuauguacauguaauuuuacagaguucaauaguuaauuauuuaauuacuuauuuucacuuuauaguuacacucuucuccccacguccuuuuuucuuauca

SEQ ID NO: 7 - DNA, 1949 bp, Candida parapsilosis
  CDS (162)..(1679)
cacacacacacacgggttagagcatctgaacttcacgacaactgttttctgtacaaccaaaacaaaccacaacacaagacaacacaacacagtactttgctgacattcaataccaatacccgtcttttttttttcaccacacaacgatattgctttgaaggatgtcacgcacttacaatgggtcttcctcctcatcaatatttctgaaaccacgtcgatttttcaataactactttctactggaaaaatcaccaacttcaacattacctgatttagaaacaaacactagcatcagaactagtcttcatagactaagacatcacaaattcataaagggagagattttacactatgggtttctttcttttgtttggttatttgtctttaccatatacccagcatccatcttgtttaaaattccagttttacttgcattgctaaccattattactattccagcaacttcacaattttttttaccagcacttcccattttgacttggttgggtttttacttcactagttccaaaatacctcattcttggaaaccagctatttcagtcaaggttttaccagccatggagactatcttgtatggtgatgatttatcaaatgtattggcaaccatcactaaccgtggtttggatattttgggctggttaccttatggtttatttcattttggtgctccatttgttgtagctgcaattatgtttttctttgctccaccaactagtttaagatcatttgcgtttgcttttggttatatgaacttgtttggggttattattcaaatgatgtttccagctgctccaccttggtacaagaatttgtatggattacaaccagccaactataccatgcatggctcaccaggtggattgggccgtatagatgaattattgggagtcgatatgtacaccactgggttctccaactcaccggttatttttggtgctttcccatcattacattcaggttgttgtattatggaggttttgtttatgtgttggttatttccccgtttgaaagtactttgggtgttttatgcatcttggttatggtggagtacaatgtacttgactcatcactattttgttgatttaactggtggtgctgtattatcattggttgtttttgaatttgtcaagtttaaatacctacctaaggctaacaagcgttgtcgttggtcttatacggaattggaatactacagcacaaccaatgatgaccctttaggaggtaacagtgctgccactcgagttgttcgcggcggcgatttggaaaattttgcatattcaagagtatacccatcattgtcacagcagcaacaattgcaagagcatccgcaacaacaacaacaatcactgtctaatcaagaaaacatacaagaggaggtgtttgaaatgggcaccatctcacgttcaagatcatcaccaaggtcctttgtttctcttggaagtagcaccaatgttgctgaagagaactttgaccttgctgatgaggaagaagatatcggttcaggatcatcaaaaactccctcagtatttgaagaagatacacaaaattacgtcgtttctcaaactacctccgttgaagacttgactaataatacacaagcatcagctgcaggcaacggagctaccgataagtcatcaagacttggaatcaatggttgaacaaagggtagatgtattagaagagtaatcgtttcttttttaatggctgcatttgtctagaagagttcgattataacgatgtttgatgatgtttatgttgatgattcacgatttttacgaattgtacaagaaccctattcacaacctgagatgggattagaatgtattgaacttggaaaagatatgggacgtgtgcttggaatgtcctccttttctcatatccactgccccattgctatccctttacaagattattaccatgttatgtct

SEQ ID NO: 8 - protein, 505 aa, Candida parapsilosis
MSRTYNGSSSSSIFSKPRRFFNNYFLSEKSPTSTLPDLETNTSIRTSLHRLRHHKFIKGEILHYGFLSFVWLFVFTIYPASILFKIPVLLALLTIITIPATSQFFLPALPILTWLGFYFTSSKIPHSWKPAISVKVLPAMETILYGDDLSNVLATITNRGLDILGWLPYGLFHFGAPFVVAAIMFFFAPPTSLRSFAFAFGYMNLFGVIIQMMFPAAPPWYKNLYGLQPANYTMHGSPGGLGRIDELLGVDMYTTGFSNSPVIFGAFPSLHSGCCIMEVLFMCWLFPRLKVLWVFYASWLWWSTMYLTHHYFVDLTGGAVLSLVVFEFVKFKYLPKANKRCRWSYTELEYYSTTNDDPLGGNSAATRVVRGGDLENFAYSRVYPSLSQQQQLQEHPQQQQQSSSNQENIQEEVFEMGTISRSRSSPRSFVSLGSSTNVAEENFDLADEEEDIGSGSSKTPSVFEEDTQNYVVSQTTSVEDLTNNTQASAAGNGATDKSSRLGING

SEQ ID NO: 9 - RNA, 1788 nt, Candida parapsilosis
augucacgcacuuacaaugggucuuccuccucaucaauauuucugaaaccacgucgauuuuucaauaacuacuuucuacuggaaaaaucaccaacuucaacauuaccugauuuagaaacaaacacuagcaucagaacuagucuucauagacuaagacaucacaaauucauaaagggagagauuuuacacuauggguuucuuucuuuuguuugguuauuugucuuuaccauauacccagcauccaucuuguuuaaaauuccaguuuuacuugcauugcuaaccauuauuacuauuccagcaacuucacaauuuuuuuuaccagcacuucccauuuugacuugguuggguuuuuacuucacuaguuccaaaauaccucauucuuggaaaccagcuauuucagucaagguuuuaccagccauggagacuaucuuguauggugaugauuuaucaaauguauuggcaaccaucacuaaccgugguuuggauauuuugggcugguuaccuuaugguuuauuucauuuuggugcuccauuuguuguagcugcaauuauguuuuucuuugcuccaccaacuaguuuaagaucauuugcguuugcuuuugguuauaugaacuuguuugggguuauuauucaaaugauguuuccagcugcuccaccuugguacaagaauuuguauggauuacaaccagccaacuauaccaugcauggcucaccagguggauugggccguauagaugaauuauugggagucgauauguacaccacuggguucuccaacucaccgguuauuuuuggugcuuucccaucauuacauucagguuguuguauuauggagguuuuguuuauguguugguuauuuccccguuugaaaguacuuuggguguuuuaugcaucuugguuaugguggaguacaauguacuugacucaucacuauuuuguugauuuaacugguggugcuguauuaucauugguuguuuuugaauuugucaaguuuaaauaccuaccuaaggcuaacaagcguugucguuggucuuauacggaauuggaauacuacagcacaaccaaugaugacccuuuaggagguaacagugcugccacucgaguuguucgcggcggcgauuuggaaaauuuugcauauucaagaguauacccaucauugucacagcagcaacaauugcaagagcauccgcaacaacaacaacaaucacugucuaaucaagaaaacauacaagaggagguguuugaaaugggcaccaucucacguucaagaucaucaccaagguccuuuguuucucuuggaaguagcaccaauguugcugaagagaacuuugaccuugcugaugaggaagaagauaucgguucaggaucaucaaaaacucccucaguauuugaagaagauacacaaaauuacgucguuucucaaacuaccuccguugaagacuugacuaauaauacacaagcaucagcugcaggcaacggagcuaccgauaagucaucaagacuuggaaucaaugguugaacaaaggguagauguauuagaagaguaaucguuucuuuuuuaauggcugcauuugucuagaagaguucgauuauaacgauguuugaugauguuuauguugaugauucacgauuuuuacgaauuguacaagaacccuauucacaaccugagaugggauuagaauguauugaacuuggaaaagauaugggacgugugcuuggaauguccuccuuuucucauauccacugccccauugcuaucccuuuacaagauuauuaccauguuaugucu

SEQ ID NO: 10 - DNA, 2214 bp, Candida tropicalis
  CDS (601)..(1974)
cgatggtgatttctttttctctcctattcctgtaccccgcaaatgtgtgatttcaatcccaaggtgccaaagcacactgatgttggcttgaaagactcagatatggagctttgtaatataagaaaaatgccaaagtgcttttcttgccaatcattgtcgatttggacttttctgcctctgtctcactttgcatctttagtgacgcatacgggaaaaaatgtaaaatgcgctgaccataaatgacattcggaacataatgcatagaatattaataccgcataccgataacagaagtcacaaacgtcacaaaacgtttcaaaaaaacagaaacaggcaagcctgaaagttgaacacagccacttcttccccaaactatgaaaacttggtcgttgcaagatcattcacttgcgcccctattttatagcatggaaaggatgcgtttctgttccggcccatttgcgcgcatttgaggtcacgtgcgctgcatcgtcacggcccattcctttttctatccttttattttatgttgcttcagccccaacgctgaaaggccgtcttttccagcagaatccattactctgctatatttccctcattccaatgtcaacggtcctaaggttcaagcctttccgcgttgcttacaagcaattccacaattattttttgctggagaaagaagcaggatgcactttggcggatcttgatttcgatactgatatccgcaagtccatccagaaactacgttcccatgagtttacgccgggcgaaatagtgcactacactttcctcacgctgttgatcttgtttgtttatgtcattttccccgcgtcgtttttgctcaaaacgcccatcttggcggctttcggcttttgtttcttattgcccttgacgtcccagtttttcgtgtcagcgttgcccattttcacatggttggcgcttttcttctctgccagcaagatccctgtggactggaagcctgtcattagcgtcaagtttcttcctgccatggaaactatcttgtacggagacaacttgtcgaatgttttggcagccaccaacaactgggtgctagatatcttggcgtggttgccctacggtatcatccactttctgtctccatttgtagttgcgctcttcattttcctttttgctccgcccacttctttgcgctcgtttggcttcgcctttggctacatgaatgtggttggtgtgttgatccagctttgtttcccctcagctgctccgtggtacaagaacttgcacggcctagaacctgcaaattattccatgaacgggtctcctggtggcttgggccgcattgacgagcttttcggtgtcgacatgtacaccactactttcatgaactcaccattggttttcggcgcgttcccctcgctccattcgggctgcgcagtcatggatgtgctctttttgtgctggctcttccctcgtttcacctatgtttggtggagctacgcctgtcttttgtggtggagcaccatgtatttgacgcaccactattttatcgacttgatcttcggtgccgctttgtctgtgctagttttcaactatgtcaagtacacgcagttgccagtggtggaccccaccaagttctgtcgctggctgtacaccaccgtggagcttcttgacattaacgccaacgacccattgcagagtttcgtgccattacacggagacatggaggaaggtatgctgcatcccttttaccacgcgccagcgcttgagaatttcgaaatgtctactataaaccgttcacgggaaagcgtggctgcgccgtttccgaccatagccgaggatgtggctgaggaaaccgacagcagcgcgtccaattcggtttttgatgctgagcgttttgacgaagaaacgcatcctagcctgcgtacgtccagcacctcgttgaacgacttgcttcttcccctgagctcgagcagctcaaagcccaaaagaagcacgtagccttttacattgtcgcaccgctctttggccaggagcactttaatattggccgtgtatactacataaatgcgacgtctatcgtgatgctatgtacattgtgagtcctagctcggtatatgatatatcctctttcgttttcgttcgtagcttccttcacttcctaatcgttccttctccttagtttgacaccttctcgaacactctttggtaagtagtgcccacattcaagttgccctt

SEQ ID NO: 11 - protein, 458 aa, Candida tropicalis
MSTVLRFKPFRVAYKQFHNYFLSEKEAGCTLADLDFDTDIRKSIQKLRSHEFTPGEIVHYTFLTLSILFVYVIFPASFLLKTPILAAFGFCFLLPLTSQFFVSALPIFTWLALFFSASKIPVDWKPVISVKFLPAMETILYGDNLSNVLAATNNWVLDILAWLPYGIIHFSSPFVVALFIFLFAPPTSLRSFGFAFGYMNVVGVLIQLCFPSAAPWYKNLHGLEPANYSMNGSPGGLGRIDELFGVDMYTTTFMNSPLVFGAFPSLHSGCAVMDVLFLCWLFPRFTYVWWSYACLLWWSTMYLTHHYFIDLIFGAALSVLVFNYVKYTQLPVVDPTKFCRWSYTTVELLDINANDPLQSFVPLHGDMEEGMSHPFYHAPALENFEMSTINRSRESVAAPFPTIAEDVAEETDSSASNSVFDAERFDEETHPSSRTSSTSLNDLLLPSSSSSSKPKRST

SEQ ID NO: 12 - RNA, 2214 nt, Candida tropicalis
cgauggugauuucuuuuucucuccuauuccuguaccccgcaaaugugugauuucaaucccaaggugccaaagcacacugauguuggcuugaaagacucagauauggagcuuuguaauauaagaaaaaugccaaagugcuuuucuugccaaucauugucgauuuggacuuuucugccucugucucacuuugcaucuuuagugacgcauacgggaaaaaauguaaaaugcgcugaccauaaaugacauucggaacauaaugcauagaauauuaauaccgcauaccgauaacagaagucacaaacgucacaaaacguuucaaaaaaacagaaacaggcaagccugaaaguugaacacagccacuucuuccccaaacuaugaaaacuuggucguugcaagaucauucacuugcgccccuauuuuauagcauggaaaggaugcguuucuguuccggcccauuugcgcgcauuugaggucacgugcgcugcaucgucacggcccauuccuuuuucuauccuuuuauuuuauguugcuucagccccaacgcugaaaggccgucuuuuccagcagaauccauuacucugcuauauuucccucauuccaaugucaacgguccuaagguucaagccuuuccgcguugcuuacaagcaauuccacaauuauuuuuugcuggagaaagaagcaggaugcacuuuggcggaucuugauuucgauacugauauccgcaaguccauccagaaacuacguucccaugaguuuacgccgggcgaaauagugcacuacacuuuccucacgcuguugaucuuguuuguuuaugucauuuuccccgcgucguuuuugcucaaaacgcccaucuuggcggcuuucggcuuuuguuucuuauugcccuugacgucccaguuuuucgugucagcguugcccauuuucacaugguuggcgcuuuucuucucugccagcaagaucccuguggacuggaagccugucauuagcgucaaguuucuuccugccauggaaacuaucuuguacggagacaacuugucgaauguuuuggcagccaccaacaacugggugcuagauaucuuggcgugguugcccuacgguaucauccacuuucugucuccauuuguaguugcgcucuucauuuuccuuuuugcuccgcccacuucuuugcgcucguuuggcuucgccuuuggcuacaugaaugugguugguguguugauccagcuuuguuuccccucagcugcuccgugguacaagaacuugcacggccuagaaccugcaaauuauuccaugaacgggucuccugguggcuugggccgcauugacgagcuuuucggugucgacauguacaccacuacuuucaugaacucaccauugguuuucggcgcguuccccucgcuccauucgggcugcgcagucauggaugugcucuuuuugugcuggcucuucccucguuucaccuauguuugguggagcuacgccugucuuuugugguggagcaccauguauuugacgcaccacuauuuuaucgacuugaucuucggugccgcuuugucugugcuaguuuucaacuaugucaaguacacgcaguugccagugguggaccccaccaaguucugucgcuggcuguacaccaccguggagcuucuugacauuaacgccaacgacccauugcagaguuucgugccauuacacggagacauggaggaagguaugcugcaucccuuuuaccacgcgccagcgcuugagaauuucgaaaugucuacuauaaaccguucacgggaaagcguggcugcgccguuuccgaccauagccgaggauguggcugaggaaaccgacagcagcgcguccaauucgguuuuugaugcugagcguuuugacgaagaaacgcauccuagccugcguacguccagcaccucguugaacgacuugcuucuuccccugagcucgagcagcucaaagcccaaaagaagcacguagccuuuuacauugucgcaccgcucuuuggccaggagcacuuuaauauuggccguguauacuacauaaaugcgacgucuaucgugaugcuauguacauugugaguccuagcucgguauaugauauauccucuuucguuuucguucguagcuuccuucacuuccuaaucguuccuucuccuuaguuugacaccuucucgaacacucuuugguaaguagugcccacauucaaguugcccuu

SEQ ID NO: 13 - DNA, 2935 bp, Aspergillus fumigatus
  CDS (450)..(1757)
attttcttccccataacaactcttctcgcccttcctccggctccgtggccaaattgttttatgcagcgcctcctagcgatttaacctcgttctcgttgcccttgcctgtccgccttgcgtcagtacgacccttgcaacgtgaccttccccagagtatcctcgtttggccgctggagaccggagcttgcaccctcataaactagctcttcgaaatcaattctccgttctccagagattatcggatcgaatctctccgctgtcgacacctttcgtctctcggtgatcctcgcccttggagtctcgtcacgttgacgccttgaacccctggccgccaactccacataggagaccacacttcattcttcccccgccataattgcagcaccctccgtctcccttcgagctcctcctggatcatcaagtccgaaaggattagactcgtcgcagcgatgaataccacccttccatcctggaaggatcggacgcaaaaccagttcggcaagctccagatccaagtcccatggcgcaccatacaacttctcgtgccgcaccgtatgcgacggaagattcggtccaagctgcgcagtcggatctcgcctacctcatcgatatcctcgttgcagacgtcattctcacctgtcgatacactcaggtcgctgcaaagtcatagatggacgctctatgactttcagtatcttttgctgctgattgtcggcatattctcgctgagcgttatggaatcacctggaccattggcaaagaccgccgcgtttacgctacttctcgtctctctccttctcccgattacgcgccagttcttcttgccattcctcccgattgcaggatggcttatatttttctacgcttgccagttcatcccgagcgactggcgccctgcaatctgggttcgcgtgctgccggctctggaaaacattctctacggtgctaatatcagtaacatcctttccgctcaccaaaatgtggtgcttgacgttttggcgtggcttccctacggaatctgccattatggcgcgccatttgtgtgctcagcgatcatgttcatctttggtcctcccggcaccgtcccccttttcgctcgaacttttggatacatcagcatggctgcagtcaccattcagctgtttttcccctgctctcctccgtggtacgaaaatctgtatggtttggctccggctgattactccatgccgggtaatcctgcgggccttgctcgcatcgatgagctttttgggatagacttgtacacatcgggcttcagacaatctcccgtcgtgtttggcgcatttccttccctacatgccgctgattcgacacttgcagctctatttatgagccaagtgttcccacggttgaagcccttgtttgtcatctatactctctggatgtggtgggctacaatgtatctttcgcaccactacgctgttgatctggtcggtggtggcctcttggcaactgtcgcgttctactttgctaaaacgcggttcatgcctcgcgtccagaatgataagatgttccgctgggactacgattatgttgagtcaggcgattccgcactcgactatgggtacggtccagccagcttcgaaggcgaattcaaccttgatagcgatgagtggaccgttggttcttcgtcatccatttcgtccggctccctcagtccagttgacgaccactactcttgggagggcgagactcttgcctctcctgccaccgagatcgagtctggaaggcatttctgatcctgctcaatgagccttgatacgtactacactgtgtacgtgctactgcattgactaatgagacggcgttttcaaacaaattttaacgacatgcttggttatcgcattgagctgatttcgacacatatatatgtttaatacgttttggggacactccagggattcatgacggttgcttcatatcccgacctggggatggattgacctggttgtgcccaattttcttctgcctaacgttttgattatacatgcatttttcacgaaaccagccggcccgccatgatcgtgacctcaatttgagctcgaatcttcctggggcctccagcgataattcttaatgctcgttccgagggtgccacatcggacattcgcttgtacaacttttgcagaacgaacattttcaccgattccaacttgagtcattcgcttactactttcaactggtcgagaaacttcgcttcttttcagctcggctaggtgcataaatattttacattcgtgtcgatcgctcacatttcacggcgcctggaaacttgggggtttcgatttcattggaaaggataaacaacatgggctgggcgccttttacacgcactacattcgcttagaaaagttctgatgcttttaatgattcttgcattggcatatagaaaggggtcctccagactcgctactgtggtcctctctcaaccccacactcgcttgcttttaacagtggacaccccgtggagctacgtctccatcaaatatttggcatcaaccggaatcgatgccaggaggactgagcttactcacggtgaccgtcgggtaaaaggcgttctaacagaacatctcctctatcctcctgtcccatctcgattctctggctgctggacgcaacacacctcgctgcacgttttcgacttcctaatacgacctaatatcattctcggttttctttgctctggctcgcggccgccatttatatggcgtgtcggctcgagtctgaagtgaacttttttctcctttctggcctccacaactttccgatccctagcagcttcccgtgcacagcgaggtgttgttggatgattgttccatagcattatcattattcctaatccggtagcgttatgatttatgaagaacagtgatgtacattattatgcggtgactaaaaaaaaaaaaaaaaaaaa

SEQ ID NO: 14 - protein, 436 aa, Aspergillus fumigatus
MNTTLPSWKDRTQNQFGKLQIQVPWRTIQLLVPHRMRRKIRSKLRSRISPTSSISSLQTSFSPVDTLRSLQSHRWTLYDFQYLLLLIVGIFSLSVMESPGPLAKTAAFTLLLVSLLLPITRQFFLPFLPIAGWLIFFYACQFIPSDWRPAIWVRVLPALENILYGANISNILSAHQNVVLDVLAWLPYGICHYGAPFVCSAIMFIFGPPGTVPLFARTFGYISMAAVTIQLFFPCSPPWYENLYGLAPADYSMPGNPAGLARIDELFGIDLYTSGFRQSPVVFGAFPSLHAADSTLAALFMSQVFPRLKPLFVIYTLWMWWATMYLSHHYAVDLVGGGLLATVAFYFAKTRFMPRVQNDKMFRWDYDYVESGDSALDYGYGPASFEGEFNLDSDEWTVGSSSSISSGSLSPVDDHYSWEGETLASPATEIESGRHF

SEQ ID NO: 15 - RNA, 2959 nt, Aspergillus fumigatus
auaugauuuaaaauuaauguuuccauuuucuuccccauaacaacucuucucgcccuuccuccggcuccguggccaaauuguuuuaugcagcgccuccuagcgauuuaaccucguucucguugcccuugccuguccgccuugcgucaguacgacccuugcaacgugaccuuccccagaguauccucguuuggccgcuggagaccggagcuugcacccucauaaacuagcucuucgaaaucaauucuccguucuccagagauuaucggaucgaaucucuccgcugucgacaccuuucgucucucggugauccucgcccuuggagucucgucacguugacgccuugaaccccuggccgccaacuccacauaggagaccacacuucauucuucccccgccauaauugcagcacccuccgucucccuucgagcuccuccuggaucaucaaguccgaaaggauuagacucgucgcagcgaugaauaccacccuuccauccuggaaggaucggacgcaaaaccaguucggcaagcuccagauccaagucccauggcgcaccauacaacuucucgugccgcaccguaugcgacggaagauucgguccaagcugcgcagucggaucucgccuaccucaucgauauccucguugcagacgucauucucaccugucgauacacucaggucgcugcaaagucauagauggacgcucuaugacuuucaguaucuuuugcugcugauugucggcauauucucgcugagcguuauggaaucaccuggaccauuggcaaagaccgccgcguuuacgcuacuucucgucucucuccuucucccgauuacgcgccaguucuucuugccauuccucccgauugcaggauggcuuauauuuuucuacgcuugccaguucaucccgagcgacuggcgcccugcaaucuggguucgcgugcugccggcucuggaaaacauucucuacggugcuaauaucaguaacauccuuuccgcucaccaaaauguggugcuugacguuuuggcguggcuucccuacggaaucugccauuauggcgcgccauuugugugcucagcgaucauguucaucuuugguccucccggcaccgucccccuuuucgcucgaacuuuuggauacaucagcauggcugcagucaccauucagcuguuuuuccccugcucuccuccgugguacgaaaaucuguaugguuuggcuccggcugauuacuccaugccggguaauccugcgggccuugcucgcaucgaugagcuuuuugggauagacuuguacacaucgggcuucagacaaucucccgucguguuuggcgcauuuccuucccuacaugccgcugauucgacacuugcagcucuauuuaugagccaaguguucccacgguugaagcccuuguuugucaucuauacucucuggauguggugggcuacaauguaucuuucgcaccacuacgcuguugaucuggucggugguggccucuuggcaacugucgcguucuacuuugcuaaaacgcgguucaugccucgcguccagaaugauaagauguuccgcugggacuacgauuauguugagucaggcgauuccgcacucgacuauggguacgguccagccagcuucgaaggcgaauucaaccuugauagcgaugaguggaccguugguucuucgucauccauuucguccggcucccucaguccaguugacgaccacuacucuugggagggcgagacucuugccucuccugccaccgagaucgagucuggaaggcauuucugauccugcucaaugagccuugauacguacuacacuguguacgugcuacugcauugacuaaugagacggcguuuucaaacaaauuuuaacgacaugcuugguuaucgcauugagcugauuucgacacauauauauguuuaauacguuuuggggacacuccagggauucaugacgguugcuucauaucccgaccuggggauggauugaccugguugugcccaauuuucuucugccuaacguuuugauuauacaugcauuuuucacgaaaccagccggcccgccaugaucgugaccucaauuugagcucgaaucuuccuggggccuccagcgauaauucuuaaugcucguuccgagggugccacaucggacauucgcuuguacaacuuuugcagaacgaacauuuucaccgauuccaacuugagucauucgcuuacuacuuucaacuggucgagaaacuucgcuucuuuucagcucggcuaggugcauaaauauuuuacauucgugucgaucgcucacauuucacggcgccuggaaacuuggggguuucgauuucauuggaaaggauaaacaacaugggcugggcgccuuuuacacgcacuacauucgcuuagaaaaguucugaugcuuuuaaugauucuugcauuggcauauagaaagggguccuccagacucgcuacugugguccucucucaaccccacacucgcuugcuuuuaacaguggacaccccguggagcuacgucuccaucaaauauuuggcaucaaccggaaucgaugccaggaggacugagcuuacucacggugaccgucggguaaaaggcguucuaacagaacaucuccucuauccuccugucccaucucgauucucuggcugcuggacgcaacacaccucgcugcacguuuucgacuuccuaauacgaccuaauaucauucucgguuuucuuugcucuggcucgcggccgccauuuauauggcgugucggcucgagucugaagugaacuuuuuucuccuuucuggccuccacaacuuuccgaucccuagcagcuucccgugcacagcgagguguuguuggaugauuguuccauagcauuaucauuauuccuaauccgguagcguuaugauuuaugaagaacagugauguacauuauuaugcggugacuaaaaaaaaaaaaaaaaaaaa

SEQ ID NO: 16 - DNA, 2856 bp, Aspergillus nidulans
  CDS (395)..(1714)
ggtttatactccggctccgtggccatctgcctccctcacgacctcctcgttccaggttttcctctcgactgctgcgcccttgcacttcgccttgcatcagtgaaaccccctgcaacgtgacggctcaaagacatcctcgtttggccgctggagaccggagcgtgcgcttcgtttcgtcttcttcgaaccgatctcaatttccccgctcgggttgacgccgtcagcaccctgctcgttgcctcccggcttgttattcaagaccccttttctgccgcttccgcgaccgatttattcgtcgccttccaactcttgtacaatcggggggaaagaaagcagacggagttcgatctggaggaattatagctgagtcttgcccgcaagactcgccgcaaccatgaatcaaacacttcccacgtggaaggaccgcacggacaaccagtttggaaagcttcagatccaggttccatggcggtccatccaactgctcgtcccgcatcgcatgcggcggaagttaaggtccaaattgcgcagtagagcgtctcctacctcgtcaatagcctctttacagacgtcgttatcgcctgcagacacactacgatcgctccaaagccaccgatggacggtttacgacttccaatatctgcttctgttgatcgtgggcatcttctctttgaccgttatcgagtcgcccgggcctttgggcaaaacggccattttctccatgctcctattctctctcctgatccctatgacccgccagttcttcctcccgtttctgccgattgccggatggcttctgtttttctacgcctgccagttcatcccaagcgattggcgccctgcgatttgggttcgtgtcttgcctgcactggagaatattctctacggcgcaaacatcagcaacatcctatccgctcaccagaacgttgtgcttgacgtgctggcgtggctaccctacggtatctgccactatggcgctccgtttgtgtgctcgttgatcatgttcatcttcggtccgcccggcactgttccccttttcgcgcgcactttcggctatatcagtatgactgcggttactattcagctgtttttcccttgctctccaccttggtatgagaatcgctatggtctagctccggcagactactccatccaaggtgatcccgcagggcttgcccgcattgacaagcttttcggcatcgacctttacacgtctgttttccatcagtcgcctgttgtgttcggcgcttttccgtcgctgcatgctgccgactcaaccctggccgcacttttcatgagtcatgttttcccccgcatgaagcccgtcttcgtgacctatactctatggatgtggtgggcaacaatgtacctctcacatcactatgcggtcgatttggttgcgggtggtctcctggccgccattgctttctacttcgccaagacccgattccttccccgtgtccagctcgacaagaccttccgttgggactacgactatgtggaattcggcgagtctgccctggagtatgggtatggtgcagctggctatgatggagacttcaatctcgacagcgatgaatggactgttggttcttcatcctccgtctcctcaggctccttgagtcccgttgacgatcattactcatgggaaaccgaggcactgacctccccacatactgatattgagtccggcaggcatactttcagcccttgagtagccacaaaccaaactcgatacctgcatatagcgatctcgctcctcctccactgcatctatttacgagacggcgttagaacatttcacgacattctggctttattgcatcgagcacatttcgacacatatatctttaataccctttcttcggtgtcccagatcatcggttcgaccttaatgtacctcggtccgaatccgcctgggatactgtttctctttccgccgcacttcactgtacattgcttgacattgcgaaaccgggttgggctcgaacgtgggatgggttatcgctcatcgctacacgccgttgctccatcataatgttaatggacacaatggggctacgcatcctggtgtttagtcctggaagaccatccgataacccccgtcggtaacactcgcttgtctcgtgtccacccagacactacttcaattctcacttctatcgtccgctattaccttgacctggtcgaacccatccttattattcgtttcgactatgctatatatttatttttaccattcgtgtcgatcgctcatactcttggcgcttgggactggaagcatttatattggaaaaaatcacggaatggggcgccttttcttcttgcacttcactcgctgtgcatagacggttttacatttctgctttgcaatgcatcacgaactctgcattagcatatagaaagaggggaaggatggaccttcttcttgattgctcgcatggtttatccattcgctcaaagtggattacgtccacatcttacccgggggctatacacatggctactgtgttgctttctgacattcgccggacgtgcaaggttgggaggagagtctgacgctgacggggcttgttgaaggatgttcacgcgtcccgatttgacccggcttcgactaacctcagattctcgacttgttggacggtgacttgacttgcttgctatggtctgacgctctcacacctacctatcacatcctcctcacctcacaaattccgctcatggacactatcctcttcttttcgtttcccttggatagtgtgtgtgtgtgtgtggttggggcaaattatccatagcagcagtattattagttataatccggtagtgttatgatttatgaaggcaacttgtatacta

SEQ ID NO: 17 - protein, 439 aa, Aspergillus nidulans
MNQTLPTWKDRTDNQFGKLQIQVPWRSIQLLVPHRMRRKLRSKLRSRASPTSSIASLQTSLSPADTLRSLQSHRWTVYDFQYLLLLIVGIFSLTVIESPGPLGKTAIFSMLLFSLLIPMTRQFFLPFLPIAGWLLFFYACQFIPSDWRPAIWVRVLPALENILYGANISNILSAHQNVVLDVLAWLPYGICHYGAPFVCSLIMFIFGPPGTVPLFARTFGYISMTAVTIQLFFPCSPPWYENRYGLAPADYSIQGDPAGLARIDKLFGIDLYTSVFHQSPVVFGAFPSLHAADSTLAALFMSHVFPRMKPVFVTYTLWMWWATMYLSHHYAVDLVAGGLLAAIAFYFAKTRFLPRVQLDKTFRWDYDYVEFGESALEYGYGAAGYDGDFNLDSDEWTVGSSSSVSSGSLSPVDDHYSWETEALTSPHTDIESGRHTFSP

SEQ ID NO: 18 - RNA, 2877 nt, Aspergillus nidulans
uaaaauuucauaaauguuuccgguuuauacuccggcuccguggccaucugccucccucacgaccuccucguuccagguuuuccucucgacugcugcgcccuugcacuucgccuugcaucagugaaacccccugcaacgugacggcucaaagacauccucguuuggccgcuggagaccggagcgugcgcuucguuucgucuucuucgaaccgaucucaauuuccccgcucggguugacgccgucagcacccugcucguugccucccggcuuguuauucaagaccccuuuucugccgcuuccgcgaccgauuuauucgucgccuuccaacucuuguacaaucggggggaaagaaagcagacggaguucgaucuggaggaauuauagcugagucuugcccgcaagacucgccgcaaccaugaaucaaacacuucccacguggaaggaccgcacggacaaccaguuuggaaagcuucagauccagguuccauggcgguccauccaacugcucgucccgcaucgcaugcggcggaaguuaagguccaaauugcgcaguagagcgucuccuaccucgucaauagccucuuuacagacgucguuaucgccugcagacacacuacgaucgcuccaaagccaccgauggacgguuuacgacuuccaauaucugcuucuguugaucgugggcaucuucucuuugaccguuaucgagucgcccgggccuuugggcaaaacggccauuuucuccaugcuccuauucucucuccugaucccuaugacccgccaguucuuccucccguuucugccgauugccggauggcuucuguuuuucuacgccugccaguucaucccaagcgauuggcgcccugcgauuuggguucgugucuugccugcacuggagaauauucucuacggcgcaaacaucagcaacauccuauccgcucaccagaacguugugcuugacgugcuggcguggcuacccuacgguaucugccacuauggcgcuccguuugugugcucguugaucauguucaucuucgguccgcccggcacuguuccccuuuucgcgcgcacuuucggcuauaucaguaugacugcgguuacuauucagcuguuuuucccuugcucuccaccuugguaugagaaucgcuauggucuagcuccggcagacuacuccauccaaggugaucccgcagggcuugcccgcauugacaagcuuuucggcaucgaccuuuacacgucuguuuuccaucagucgccuguuguguucggcgcuuuuccgucgcugcaugcugccgacucaacccuggccgcacuuuucaugagucauguuuucccccgcaugaagcccgucuucgugaccuauacucuauggauguggugggcaacaauguaccucucacaucacuaugcggucgauuugguugcggguggucuccuggccgccauugcuuucuacuucgccaagacccgauuccuuccccguguccagcucgacaagaccuuccguugggacuacgacuauguggaauucggcgagucugcccuggaguauggguauggugcagcuggcuaugauggagacuucaaucucgacagcgaugaauggacuguugguucuucauccuccgucuccucaggcuccuugagucccguugacgaucauuacucaugggaaaccgaggcacugaccuccccacauacugauauugaguccggcaggcauacuuucagcccuugaguagccacaaaccaaacucgauaccugcauauagcgaucucgcuccuccuccacugcaucuauuuacgagacggcguuagaacauuucacgacauucuggcuuuauugcaucgagcacauuucgacacauauaucuuuaauacccuuucuucggugucccagaucaucgguucgaccuuaauguaccucgguccgaauccgccugggauacuguuucucuuuccgccgcacuucacuguacauugcuugacauugcgaaaccggguugggcucgaacgugggauggguuaucgcucaucgcuacacgccguugcuccaucauaauguuaauggacacaauggggcuacgcauccugguguuuaguccuggaagaccauccgauaacccccgucgguaacacucgcuugucucguguccacccagacacuacuucaauucucacuucuaucguccgcuauuaccuugaccuggucgaacccauccuuauuauucguuucgacuaugcuauauauuuauuuuuaccauucgugucgaucgcucauacucuuggcgcuugggacuggaagcauuuauauuggaaaaaaucacggaauggggcgccuuuucuucuugcacuucacucgcugugcauagacgguuuuacauuucugcuuugcaaugcaucacgaacucugcauuagcauauagaaagaggggaaggauggaccuucuucuugauugcucgcaugguuuauccauucgcucaaaguggauuacguccacaucuuacccgggggcuauacacauggcuacuguguugcuuucugacauucgccggacgugcaagguugggaggagagucugacgcugacggggcuuguugaaggauguucacgcgucccgauuugacccggcuucgacuaaccucagauucucgacuuguuggacggugacuugacuugcuugcuauggucugacgcucucacaccuaccuaucacauccuccucaccucacaaauuccgcucauggacacuauccucuucuuuucguuucccuuggauagugugugugugugugugguuggggcaaauuauccauagcagcaguauuauuaguuauaauccgguaguguuaugauuuaugaaggcaacuuguauacua

SEQ ID NO: 19 - DNA, 3220 bp, Candida neoformans
  intron (1888)..(1939)
gtcgacatagttacactcccctctactcttcagactaccccttctgattatcaggagactacgactactccgcctaccttgacctttgcaaactcggagccacgaccgagtccttccctccatcgttccctgcgcaacatcggtttggccgacggcagcatgacttgtgccccaggctatatcctcatctgcattcatcatgaccccttgtctcgttacacgcttggcatcaattttaccacctttgtgggtctttgccccacgacccctgctagtaccagcacttccggcggtcccgccttttcctcttttacacccaagttcgccgaccatatgcgatgtcatgtcatccagcgcttgctgcacactgtcatgcacgaccgtctcgccaacatgccgggttgccacacctgggaatcgttggtcaatgcggcgtaccgttactgcacctgggctatggaatgtatcttcctcggcaccgatgcaaatgttcaattcgggattgcttgttatccccacttgaaagagctcgaagaggcggcaaggacgctcaggaaggatagtatgggcgagtgggatgatgtggtgcagattggggtggataggttgttggaaatactaccttgtggtgaacggaccttgatcaacaagagaaggatcgaaaaggaacgactgcaagcgcttctaaaagctcaaactcaagtcaaggccaagcatgaggccagatgtcaggtggcaaccccgatcccagctccagctgtcattcctgtctctgcacctgctccggttataatcaagcgtaaaaaatacagtagcctacgcaagctgatgggacgtgcggggtcggtgattggcaggcagaagatgcgggcaagggcgtctgcgattatcgagaagggaaaggccggcttctacgataggcaaaaggtccagcgcatggagccggctccatccatgtctcgtcctgcgggagtgtcttgtgtgtgattgtcagctggccgtgaagatagtcttggagctgtgtatagggatactacatgatgtgcatgcagtgtgttttcaaagcaattgccacgtggtgttcgcgtgggtcggaaacgaggttcgcgttgttatggttcttgttttgagattctcgcgctggctacacgtacgcaccatacactgcagacgacagcggcaagcatacagacggcaacgctgacatcccagccacacacaccatgtccgccatccgcgcactcacgagccccgtcaccgcctgcttctccacctctctctccccgcatgccgcattccaccgcttcctccatgcgctctccgcctccatccgccgcctcgacctctcccgcgacccgcgcaagaccttggaccgcctcaggcagcacaggttcacccttgccaacactttgccccgcgcgttcatgcttctgtgcgcctcctacagtctctacatcatgaccaccccgcccttcccactcaagctcgggatacccatcgcatacatcgccgctgtcatcttcccgatcacgtcgcagtttgtctggccggcgacacccatctttgcatggctcatcacctttttctccgcccgtttcatcccttccggccgccggcccgaaatccacgtcgcccttctcccagcccttgaatccgtcctctacggtgccaacatttctgatctccaaacacgatataccaacgctttccttgatgtggtcgcctggctgccctatggggtattacatttcacccttccattcgtcgtggctgttatcctttggtcactgggacctaggggtgcggttcagttctggggtttggcctttgggtggatgaatttgctcggtgtcgtctgccagctcctcttccctgctgccgcgccttgtaagtacctctctcttttatttaatatatccatacttaatatgccgggcagggtacgaaatcattcacggtcttacccccgccgactattccatggctggctctcccggcggtctcatgcgtatcgaccgcgtcttccattcttcaggctataccaacgcattcggttccgctcctcttgtttttggcgccttcccctccctccattcaggatgtgccgtcatggaagccctcttcctctcccatttcttcccatccctcaagggcctgtattggggctatgtcggtgtcctctggtgggcgaccatgtacctctcccaccactacctcatcgacctcgttggcggagcttgtctgagcgtcttggtgttctacttgtgcatgcctgaaggtttcaaggacgttgatcagatacagtgggaggcggtggaaggggatggatacgagatgattggcggacccagaacaggcacaggtcctgagattgatttggatgaggaaattagaaagttggaagaacaaggcgaagcgctttttgagcaggtgataggggatgaagagtctcggatcgagacaaggggagaaggaaacagtggcgcgggcgggaatctgagtgggaatgaaagtggtgatagtgcagatagcggtaggggaaagggcaaggggaaggggaaggggaaacaaactgccaaaaagccaaaggcgaaggagcaaagatcggttagctggggagaaacaaaggtgatgggtgaaggagcgcaggtggcttcagagaatagctcttagatatctgttttaaaacagtagtatacccaacaattttttgacgatataccctaccactatgatcttaataatgcagaatagaaaaacaccttgcccctctcgcaggcgtcaacacatggagtctaggcagttaaatcacactacatcatcatcgccgaaaaaaataaacctagtaactccaacccaaccctcctggtacaggcctgttcgtccaccgactgataattcccatcgccctcgccctcctcacaagtttacccacccgcctttgcgacttccaagtcaacccggtctccgcccgacttcttatcttgcccatcgggttgacgaattcataggcgaaattagggttcatatcgtgcaccaaggggttcgttttggtgatgtaaaaaggatcaagcttgacggcgattttcttgggaggaccaagaagaggtggacggggcaaaggtcgagcggtggggtagatggattcttgggtgaatgcttgaggggcagtaaactagaagagttgagaaaacccaaaggagtcagcctttatgtcgac

SEQ ID NO: 20 - DNA, 3120 bp, Cryptococcus neoformans
  CDS (1224)..(2624)
gtcgacatagttacactcccctctactcttcagactaccccttctgattatcaggagactacgactactccgcctaccttgacctttgcaaactcggagccacgaccgagtccttccctccatcgttccctgcgcaacatcggtttggccgacggcagcatgacttgtgccccaggctatatcctcatctgcattcatcatgaccccttgtctcgttacacgcttggcatcaattttaccacctttgtgggtctttgccccacgacccctgctagtaccagcacttccggcggtcccgccttttcctcttttacacccaagttcgccgaccatatgcgatgtcatgtcatccagcgcttgctgcacactgtcatgcacgaccgtctcgccaacatgccgggttgccacacctgggaatcgttggtcaatgcggcgtaccgttactgcacctgggctatggaatgtatcttcctcggcaccgatgcaaatgttcaattcgggattgcttgttatccccacttgaaagagctcgaagaggcggcaaggacgctcaggaaggatagtatgggcgagtgggatgatgtggtgcagattggggtggataggttgttggaaatactaccttgtggtgaacggaccttgatcaacaagagaaggatcgaaaaggaacgactgcaagcgcttctaaaagctcaaactcaagtcaaggccaagcatgaggccagatgtcaggtggcaaccccgatcccagctccagctgtcattcctgtctctgcacctgctccggttataatcaagcgtaaaaaatacagtagcctacgcaagctgatgggacgtgcggggtcggtgattggcaggcagaagatgcgggcaagggcgtctgcgattatcgagaagggaaaggccggcttctacgataggcaaaaggtccagcgcatggagccggctccatccatgtctcgtcctgcgggagtgtcttgtgtgtgattgtcagctggccgtgaagatagtcttggagctgtgtatagggatactacatgatgtgcatgcagtgtgttttcaaagcaattgccacgtggtgttcgcgtgggtcggaaacgaggttcgcgttgttatggttcttgttttgagattctcgcgctggctacacgtacgcaccatacactgcagacgacagcggcaagcatacagacggcaacgctgacatcccagccacacacaccatgtccgccatccgcgcactcacgagccccgtcaccgcctgcttctccacctctctctccccgcatgccgcattccaccgcttcctccatgcgctctccgcctccatccgccgcctcgacctctcccgcgacccgcgcaagaccttggaccgcctcaggcagcacaggttcacccttgccaacactttgccccgcgcgttcatgcttctgtgcgcctcctacagtctctacatcatgaccaccccgcccttcccactcaagctcgggatacccatcgcatacatcgccgctgtcatcttcccgatcacgtcgcagtccgcccgtttcatcccttccggccgccggcccgaaatccacgtcgcccttctcccagcccttgaatccgtcctctacggtgccaacatttctgatctccaaacacgatataccaacgctttccttgatgtggtcgcctggctgccctatggggtattacatttcacccttccattcgtcgtggctgttatcctttggtcactgggacctaggggtgcggttcagttctggggtttggcctttgggtggatgaatttgctcggtgtcgtctgccagctcctcttccctgctgccgcgccttggtacgaaatcattcacggtcttacccccgccgactattccatggctggctctcccggcggtctcatgcgtatcgaccgcgtcttccattcttcaggctataccaacgcattcggttccgctcctcttgtttttggcgccttcccctccctccattcaggatgtgccgtcatggaagccctcttcctctcccatttcttcccatccctcaagggcctgtattggggctatgtcggtgtcctctggtgggcgaccatgtacctctcccaccactacctcatcgacctcgttggcggagcttgtctgagcgtcttggtgttctacttgtgcatgcctgaaggtttcaaggacgttgatcagatacagtgggaggcggtggaaggggatggatacgagatgattggcggacccagaacaggcacaggtcctgagattgatttggatgaggaaattagaaagttggaagaacaaggcgaagcgctttttgagcaggtgataggggatgaagagtctcggatcgagacaaggggagaaggaaacagtggcgcgggcgggaatctgagtgggaatgaaagtggtgatagtgcagatagcggtaggggaaagggcaaggggaaggggaaggggaaacaaactgccaaaaagccaaaggcgaaggagcaaagatcggttagctggggagaaacaaaggtgatgggtgaaggagcgcaggtggcttcagagaatagctcttagatatctgttttaaaacagtagtatacccaacaattttttgacgatataccctaccactatgatcttaataatgcagaatagaaaaacaccttgcccctctcgcaggcgtcaacacatggagtctaggcagttaaatcacactacatcatcatcgccgaaaaaaataaacctagtaactccaacccaaccctcctggtacaggcctgttcgtccaccgactgataattcccatcgccctcgccctcctcacaagtttacccacccgcctttgcgacttccaagtcaacccggtctccgcccgacttcttatcttgcccatcgggttgacgaattcataggcgaaattagggttcatatcgtgcaccaaggggttcgttttggtgatgtaaaaaggatcaagcttgacggcgattttcttgggaggaccaagaagaggtggacggggcaaaggtcgagcggtggggtagatggattcttgggtgaatgcttgaggggcagtaaactagaagagttgagaaaacccaaaggagtcagcctttatgtcgac

SEQ ID NO: 21 - protein, 449 aa, Cryptococcus neoformans
MSAIRALTSPVTACFSTSLSPHAAFHRFLHALSASIRRLDLSRDPRKTLDRLRQHRFTLANTLPRAFMLLCASYSLYIMTTPPFPLKLGIPIAYIAAVIFPITSQSARFIPSGRRPEIHVALLPALESVLYGANISDLQTRYTNAFLDVVAWLPYGVLHFTLPFVVAVILWSLGPRGAVQFWGLAFGWMNLLGVVCQLLFPAAAPWYEIIHGLTPADYSMAGSPGGLMRIDRVFHSSGYTNAFGSAPLVFGAFPSLHSGCAVMEALFLSHFFPSLKGLYWGYVGVLWWATMYLSHHYLIDLVGGACLSVLVFYLCMPEGFKDVDQIQWEAVEGDGYEMIGGPRTGTGPEIDLDEEIRKLEEQGEALFEQVIGDEESRIETRGEGNSGAGGNLSGNESGDSADSGRGKGKGKGKGKQTAKKPKAKEQRSVSWGETKVMGEGAQVASENSS

SEQ ID NO: 22 - protein, 6 aa, Cryptococcus neoformans
ISVLKQ

SEQ ID NO: 23 - protein, 10 aa, Cryptococcus neoformans
YTQQFFDDIP

SEQ ID NO: 24 - RNA, 3168 nt, Candida neoformans
gucgacauaguuacacuccccucuacucuucagacuaccccuucugauuaucaggagacuacgacuacuccgccuaccuugaccuuugcaaacucggagccacgaccgaguccuucccuccaucguucccugcgcaacaucgguuuggccgacggcagcaugacuugugccccaggcuauauccucaucugcauucaucaugaccccuugucucguuacacgcuuggcaucaauuuuaccaccuuugugggucuuugccccacgaccccugcuaguaccagcacuuccggcggucccgccuuuuccucuuuuacacccaaguucgccgaccauaugcgaugucaugucauccagcgcuugcugcacacugucaugcacgaccgucucgccaacaugccggguugccacaccugggaaucguuggucaaugcggcguaccguuacugcaccugggcuauggaauguaucuuccucggcaccgaugcaaauguucaauucgggauugcuuguuauccccacuugaaagagcucgaagaggcggcaaggacgcucaggaaggauaguaugggcgagugggaugauguggugcagauugggguggauagguuguuggaaauacuaccuuguggugaacggaccuugaucaacaagagaaggaucgaaaaggaacgacugcaagcgcuucuaaaagcucaaacucaagucaaggccaagcaugaggccagaugucagguggcaaccccgaucccagcuccagcugucauuccugucucugcaccugcuccgguuauaaucaagcguaaaaaauacaguagccuacgcaagcugaugggacgugcggggucggugauuggcaggcagaagaugcgggcaagggcgucugcgauuaucgagaagggaaaggccggcuucuacgauaggcaaaagguccagcgcauggagccggcuccauccaugucucguccugcgggagugucuugugugugauugucagcuggccgugaagauagucuuggagcuguguauagggauacuacaugaugugcaugcaguguguuuucaaagcaauugccacgugguguucgcgugggucggaaacgagguucgcguuguuaugguucuuguuuugagauucucgcgcuggcuacacguacgcaccauacacugcagacgacagcggcaagcauacagacggcaacgcugacaucccagccacacacaccauguccgccauccgcgcacucacgagccccgucaccgccugcuucuccaccucucucuccccgcaugccgcauuccaccgcuuccuccaugcgcucuccgccuccauccgccgccucgaccucucccgcgacccgcgcaagaccuuggaccgccucaggcagcacagguucacccuugccaacacuuugccccgcgcguucaugcuucugugcgccuccuacagucucuacaucaugaccaccccgcccuucccacucaagcucgggauacccaucgcauacaucgccgcugucaucuucccgaucacgucgcaguuugucuggccggcgacacccaucuuugcauggcucaucaccuuuuucuccgcccguuucaucccuuccggccgccggcccgaaauccacgucgcccuucucccagcccuugaauccguccucuacggugccaacauuucugaucuccaaacacgauauaccaacgcuuuccuugauguggucgccuggcugcccuaugggguauuacauuucacccuuccauucgucguggcuguuauccuuuggucacugggaccuaggggugcgguucaguucugggguuuggccuuuggguggaugaauuugcucggugucgucugccagcuccucuucccugcugccgcgccuugguacgaaaucauucacggucuuacccccgccgacuauuccauggcuggcucucccggcggucucaugcguaucgaccgcgucuuccauucuucaggcuauaccaacgcauucgguuccgcuccucuuguuuuuggcgccuuccccucccuccauucaggaugugccgucauggaagcccucuuccucucccauuucuucccaucccucaagggccuguauuggggcuaugucgguguccucuggugggcgaccauguaccucucccaccacuaccucaucgaccucguuggcggagcuugucugagcgucuugguguucuacuugugcaugccugaagguuucaaggacguugaucagauacagugggaggcgguggaaggggauggauacgagaugauuggcggacccagaacaggcacagguccugagauugauuuggaugaggaaauuagaaaguuggaagaacaaggcgaagcgcuuuuugagcaggugauaggggaugaagagucucggaucgagacaaggggagaaggaaacaguggcgcgggcgggaaucugagugggaaugaaaguggugauagugcagauagcgguaggggaaagggcaaggggaaggggaaggggaaacaaacugccaaaaagccaaaggcgaaggagcaaagaucgguuagcuggggagaaacaaaggugaugggugaaggagcgcagguggcuucagagaauagcucuuagauaucuguuuuaaaacaguaguauacccaacaauuuuuugacgauauacccuaccacuaugaucuuaauaaugcagaauagaaaaacaccuugccccucucgcaggcgucaacacauggagucuaggcaguuaaaucacacuacaucaucaucgccgaaaaaaauaaaccuaguaacuccaacccaacccuccugguacaggccuguucguccaccgacugauaauucccaucgcccucgcccuccucacaaguuuacccacccgccuuugcgacuuccaagucaacccggucuccgcccgacuucuuaucuugcccaucggguugacgaauucauaggcgaaauuaggguucauaucgugcaccaagggguucguuuuggugauguaaaaaggaucaagcuugacggcgauuuucuugggaggaccaagaagagguggacggggcaaaggucgagcggugggguagauggauucuugggugaaugcuugaggggcaguaaacuagaagaguugagaaaacccaaaggagucagccuuuaugucgac